(19) United States
(12) Patent Application Publication
(10) Pub. No.: US 2026/0297649 A1
(43) Pub. Date: Oct. 1, 2026

(54) Modification of Pseudouridine

(71) Applicant: Ludwig Institute for Cancer Research Ltd, Zurich (CH)

(72) Inventors: Haiqi Xu, Oxford (GB); Chun-Xiao Song, Oxford (GB)

(73) Assignee: Ludwig Institute for Cancer Research Ltd, Zurich (CH)

(21) Appl. No.: 19/478,205

(22) PCT Filed: Apr. 26, 2024
(86) PCT No.: PCT/EP24/61698

§ 371 Date: Oct. 23, 2025

(30) Foreign Application Priority Data
Apr. 28, 2023 (GB) 2306294.6
Nov. 28, 2023 (GB) 2318116.7

(51) Int. Cl. C12Q 1/6806; C12Q 1/48; C12Q 1/6844; C12Q 1/6869

(52) U.S. Cl. CPC C12Q 1/6806; C12Q 1/485; C12Q 1/6844; C12Q 1/6869

(57) ABSTRACT

A 2-bromoacrylamide-assisted cyclization sequencing (BACS) method permits quantitative profiling of pseudouridine (Ψ) at single-base resolution. Based on the bromoacrylamide cyclization chemistry, BACS induces Ψ-to-C mutation rather than truncation or deletion signatures during reverse transcription (RT), therefore providing higher resolution and enabling more accurate quantification of Ψ stoichiometry compared with CMC- and BS-based methods. Compared to known methods, BACS of the invention allows for the precise identification of Ψ positions, especially in densely modified Ψ regions and consecutive uridine sequences. Methods, compositions and kits are therefore possible for the detection of Ψ. In the context of RNA molecules, this allows for detection and sequencing of Ψ in such sequences.

### FIELD OF INVENTION

This invention relates to the field of molecular biology and more particularly to methods, compositions and kits for modifying, detecting, locating and otherwise determining the presence of pseudouridine in ribonucleic acid sequences.

### BACKGROUND

Pseudouridine (ψ), sometimes referred to as pseudouracil, is the C—C glycoside isomer of uridine (U), and is the most abundant post-transcriptional modification in cellular RNA. ψ is prevalent in nearly all kinds of non-coding RNA (ncRNA), including ribosomal RNA (rRNA), transfer RNA (tRNA) and small nuclear RNA (snRNA). It is also known to be present in messenger RNA (mRNA).

ψ has been revealed to play an important role in splicing, translation, RNA stability, and RNA-protein interactions. In eukaryotes, ψ is installed by various pseudouridine synthase (PUS) enzymes, which have been shown to associate with many diseases including cancer. Therefore, establishing an accurate and sensitive method to detect ψ is highly desirable.

In the ribosome, ψ residues are clustered and have the effect of stabilizing RNA-RNA and/or RNA-protein interactions. Such stability may assist in the folding of rRNA and assemble of the ribosome. The presence of ψ in rRNA can affect stability in structures nearby and thereby impact the speed and accuracy of decoding and proofreading in the process of translation.

In snRNA, ψ residues ensure proper folding and assembly of the spliceosome which is required in pre-mRNA processing.

In tRNA, the presence of ψ stabilizes stem loop structures in a way which the normal uracil residue does not.

In mRNA, ψ is the second most abundant internal modification (0.1-0.4% ψ/U ratio as measured by mass spectrometry). The presence of ψ residues in mRNA is known for example to affect the coding specificity of stop codons UAA, UGA and UAG and such modification of U to ψ leads to nonsense suppression.

ψ is formed in RNA structures post-translation and this is achieved by various kinds of ψ synthase (PUS) enzymes in eukaryotes. These enzymes may play an important role in mRNA processing, stability, and translation.

Certain genetic mutants which lack ψ residues in tRNA or rRNA have been found with difficulties in translation, causing slow growth rates in cells compared to wild-type. ψ modification defect have been found to correspond to certain diseases, such as for example, dyskeratosis congenita, mitochondrial myopathy and sideroblastic anemia (MLASA).

ψs are also known as having some function in regulation of latency in human immunodeficiency virus (HIV) infections.

Pseudouridylation is also known in connection with maternally inherited diabetes and deafness (MIDD). A point mutation in a mitochondrial tRNA may negate pseudouridylation of a nucleotide resulting in a change in tRNA structure, and thus instability leading to poor mitochondrial translation and respiration.

ψ in mRNA may also be associated with various types of cancer and other diseases and could potentially serve as a biomarker for early cancer detection.

Therefore, accurate and sensitive methods are needed to detect ψ in RNA molecules in many areas of biological and medical research. Traditionally, the detection of ψ has relied heavily on N-cyclohexyl-N′-(2-morpholinoethyl)carbodiimide methyl-p-toluenesulfonate (CMC) chemistry (Bakin, A. V. & Ofengand, J. Methods Mol. Biol. 77, 297-309 (1998)). CMC can readily react with amide or imide functional group in a nucleobase (for example, amide for guanosine and imide for uridine—Gilham, P. T. J. Am. Chem. Soc. 84, 687-688 (1962) and Ho, N. W. Y. & Gilham, P. T. Biochemistry 6, 3632-3639 (1967)), while it can form a more stable adduct with N³ of ψ, therefore enabling discrimination of ψ with U through subsequent alkaline treatment (pH ~10.4) (Ho, N. W. Y. & Gilham, P. T. Biochemistry 10, 3651-3657 (1971)). Since N³-CMC adduct of ψ significantly interferes with base pairing on the Watson-Crick side, it would result in truncation signatures in reverse transcription (RT). With the help of these RT stops, the CMC chemistry has been widely applied to transcriptional-wide sequencing of ψ, as shown in ψ-seq (Carlile, T. M. et al. Nature 515, 143-146 (2014)), Pseudo-seq (Schwartz, S. et al. Cell 159, 148-162 (2014)) and PSI-seq (Lovejoy, A. F., et al., PLoS One 9, e110799 (2014)).

However, CMC-based methods had low labelling efficiency and selectivity of ψ, making it intrinsically difficult to distinguish between true ψ signals and background noises from other bases and RNA secondary structures (see Incarnato, D., et al., Genome Biol. 15, 491 (2014) and Wang, P. Y., et al., RNA 25, 135-146 (2019). Only about 100-400 and about 50-100 ψ sites were detected on human and yeast mRNA, respectively.

Utilizing azide-CMC to enrich the truncation signals, CeU-seq could detect about 1000-2000 ψ sites in human transcriptome. Nevertheless, according to mass spectrometry results, there appear to be many more ψ sites than are actually being detected. So although CeU-seq utilized azide-labeled CMC to enrich the truncation signals and therefore increase the sensitivity, the method still suffers from partial reactivity and harsh alkaline treatment of the CMC chemistry (Li, X. et al., Nat. Chem. Biol. 11, 592-597 (2015). Therefore, a fundamental drawback of CMC-based methods may arise because of lower selectivity of CMC for ψ, making it intrinsically more difficult to distinguish true ψ signals from background. Furthermore, a relatively large amount of starting material (about 5-10 μg) is needed when using CMC-based methods, possibly due to the unavoidable RNA degradation caused by harsh alkaline treatment. Basically, all CMC-based methods lack stoichiometry information of ψ.

Recently, bisulfite (BS) treatment has been used for cytosine modification detection (Singhal, R. P. Biochemistry 13, 2924-2932 (1974), and Everett, D. W. Part I: Reaction of pseudouridine with bisulfite. Part II: Reaction of glyoxal with guanine derivatives: A spectrophotometric probe of molecular structure. (New York University, 1980)). BS treatment has been surprisingly found to convert ψ into a ψ-BS adduct and could finally lead to deletion signatures in RT (RBS-seq), thus providing an improvement over using CMC (see Khoddami, V. et al., Proc. Natl. Acad. Sci. U.S.A 116, 6784-6789 (2019); also Fleming, A. M. et al., J. Am. Chem. Soc. 141, 16450-16460 (2019)). Since unmodified cytosine (C) would also be deaminated to U in conventional BS reaction (see Shapiro, R., et al., J. Am. Chem. Soc. 92, 422-424 (1970) and Hayatsu, H., et al., J. Am. Chem. Soc. 92, 724-726 (1970)), BID-seq and pseudouridine assessment via 19 bisulfite/sulfite treatment (PRAISE) further optimized BS treatment to near neutral pH to eliminate most of the side reaction on C, enabling quantitative detection of Ψ across transcriptome (see Dai, Q. et al., Nat. Biotechnol. 41, 344-354 (2023) and Zhang, M. et al., Nat. Chem. Biol. (2023)).

WO2022/232795 THE UNIVERSITY OF CHICAGO discloses the method of modifying ψ comprising modified bisulfite treatment. As explained therein, careful examination of Ψ reactivity with bisulfite allowed for a modified reaction of DNA or DNA with bisulfite in the pH range 6.8-7.2, achieving quantitative ψ-BS formation and no C to U conversion. Related thereto is the corresponding scientific publication of Dai Q. et al (2022) Nature Biotechnology 27 Oct. 2022 DOI https://doi.org/10.10.8/s1587-22-01505-w.

Zhang M. et al. (2022) bioRxiv doi: https://doi.org/10.1101/22125.513650 describes the PRAISE method which relies on bisulfite-induced deletion signature during reverse transcription, thus enabling quantitative pseudouridine assessment via bisulfite/sulfite treatment. PRAISE is based on quaternary reads alignment and thus accurately measures ψ stoichiometry in spike-in RNA and as well as rRNA.

Although the aforementioned BS-based methods can detect about 1000-2000 ψ sites on human mRNA, they still suffer from low deletion rates and high false-positive rates in some sequence contexts. In addition, due to the deletion signature, it is difficult to determine the exact ψ site when it is adjacent to one or more U or detect consecutive ψ sites. Besides, detecting low-modified ψ sites and ψ sites in low-expressed RNA may be laborious, because sufficient coverage may need to be generated.

An improved method of pseudouridine detection and sequencing is needed and would be of use in all areas of cell biology.

### BRIEF SUMMARY OF THE DISCLOSURE

In accordance with the present invention there are provided methods, compositions and kits for the modification and detection of pseudouridine; and in the circumstance of the pseudouridine forming part of an RNA molecule, then the modification, detection and sequencing of pseudouridine residues in RNA molecules. The invention therefore provides a 2-bromoacrylamide-assisted cyclization sequencing (BACS) method for quantitative profiling of ψ at single-base resolution. Based on this bromoacrylamide cyclization chemistry, BACS induces ψ-to-C mutation rather than truncation or deletion signatures during reverse transcription (RT), therefore providing higher resolution and enabling more accurate quantification of ψ stoichiometry compared with CMC- and BS-based methods. Therefore, compared to known methods, BACS of the invention allows for the precise identification of ψ positions, especially in densely modified ψ regions and consecutive uridine sequences.

In a first aspect, the invention provides a method of modifying pseudouridine comprising reacting the pseudouridine with a Michael Addition acceptor, wherein the Michael Addition acceptor is a compound according to Formula (I):

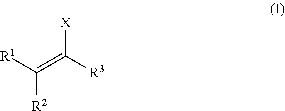

wherein:
   X is independently selected from halo, tosyl, mesyl, and triflyl;
   R¹ is independently selected from H, C₁-C₄-alkyl, C₁-C₄-haloalkyl, and C₀-C₄-alkylene-R^{1a}, wherein R^{1a} is independently selected from C₃-C₆ cycloalkyl, phenyl, and 4- to 6-membered heterocyclyl; wherein where R^{1a} is C₃-C₆ cycloalkyl or 4- to 6-membered heterocyclyl, R^{1a} is optionally substituted with from 1 to 4 R^{1b}, and where R^{1a} is phenyl, R^{1a} is optionally substituted with from 1 to 4 R^{1c};
   R² is independently selected from H, C₁-C₄-alkyl, C₁-C₄-haloalkyl, and C₀-C₄-alkylene-R^{2a}, wherein R^{2a} is independently selected from C₃-C₆ cycloalkyl, phenyl, and 4- to 6-membered heterocyclyl; wherein where R^{2a} is C₃-C₆ cycloalkyl or 4- to 6-membered heterocyclyl, R^{2a} is optionally substituted with from 1 to 4 R^{2b}, and where R^{2a} is phenyl, R^{2a} is optionally substituted with from 1 to 4 R^{2c};
   R³ is independently selected from —C(O)OR^{3a}, —C(O)R^{3a}, —C(O)NR^{3b}R^{3b}, —CN, —NO₂, —S(O)₂OR^{3a}, —S(O)₂R^{3a}, —S(O)₂NR^{3b}R^{3b}, and 5- to 10-membered heteroaryl, wherein where R³ is 5- to 10-membered heteroaryl, R³ is optionally substituted with from 1 to 4 R^{3e};
   R^{3a} is independently selected from H, C₁-C₆-alkyl, C₁-C₆-haloalkyl, and C₀-C₆-alkylene-R^{3c}; wherein where R^{3a} is alkyl or haloalkyl, R^{3a} is optionally substituted with a group selected from —N₃, —C≡CH, Dibenzocyclooctynol (DIBO), Aza-dibenzocyclooctyne (DBCO), Bicyclononyne (BCN), trans-Cyclooctene (TCO), and Tetrazine;
   R^{3b} is independently selected from H, C₁-C₆-alkyl, C₁-C₆-haloalkyl, and C₀-C₆-alkylene-R^{3c}; wherein where R^{3b} is alkyl or haloalkyl, R^{3b} is optionally substituted with a group selected from —N₃, —C≡CH, Dibenzocyclooctynol (DIBO), Aza-dibenzocyclooctyne (DBCO), Bicyclononyne (BCN), trans-Cyclooctene (TCO), and Tetrazine;
   or wherein two R^{3b} groups, together with the nitrogen atom to which they are attached form a 5- or 6-membered heterocycloalkyl group, optionally substituted with from 1 to 4 R^{3d};
   R^{3c} is independently selected from C₃—C cycloalkyl, phenyl, and 4- to 6-membered heterocyclyl; wherein where R^{3c} is C₃—C cycloalkyl or 4- to 6-membered heterocyclyl, R^{3c} is optionally substituted with from 1 to 4 R^{3d}, and where R^{3c} is phenyl, R^{3c} is optionally substituted with from 1 to 4 R^{3e}
   R^{1b} and R^{2b} are each independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
   R^{1c} and R^{2c} are each independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
   R^{3d} is independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, C₁-C₄-haloalkyl, —N₃, —C≡CH, Dibenzocyclooctynol (DIBO), Aza-dibenzocyclooctyne (DBCO), Bicyclononyne (BCN), trans-Cyclooctene (TCO), and Tetrazine;
   R^{3e} is independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl, —N₃, —C≡CH, Dibenzocyclooctynol (DIBO), Aza-dibenzocyclooctyne (DBCO), Bicyclononyne (BCN), trans-Cyclooctene (TCO), and Tetrazine;
   R⁴ is independently at each occurrence selected from H and C₁-C₄-alkyl; or where two R⁴ groups are attached to the same nitrogen, those two R⁴ groups together with the nitrogen atom to which they are attached optionally form a 5- to 6-membered-heterocycloalkyl group optionally substituted with from 1 to 4 R⁵; and
   R⁵ are each independently at each occurrence selected from ═O, ═S, halo, nitro, cyano, C₁-C₄-alkyl, and C₁-C₄-haloalkyl.

Reactions in accordance with the invention advantageously modify pseudouridine in a substantially single step and so may permit the convenient and efficient modification of pseudouridine into a cyclised derivative, particularly when comprised within an RNA or any other molecule. The cyclised derivative having differing molecular weight and chemical properties can then be used as a proxy for the identification of the presence of pseudouridine prior to the reaction taking place. In certain aspects, the modified pseudouridine includes a reactive moiety that is able to react with other molecules, as described hereinafter, such as a linker, affinity tag, imaging probe or fluorophore. Such reactive moieties may be of the types used in click chemistry, such as azide, alkyne, dibenzocyclooctynol (DIBO), aza-dibenzocyclooctyne (DBCO), bicyclononynes (BCN), trans-cyclooctenes (TNO) or tetrazine. As will be appreciated, a second reaction step may be required to link the modified pseudouridine to the further molecule, such as a linker, affinity tag, imaging probe or fluorophore.

In another aspect, the invention provides a method of tagging or labelling pseudouridine in a sample, comprising reacting at least a portion of the sample with a Michael Addition acceptor, wherein the Michael Addition acceptor is a compound according to Formula (I):

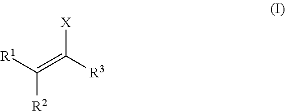

wherein:
   X is independently selected from halo, tosyl, mesyl and triflyl;
   R¹ is independently selected from H, C₁-C₄-alkyl, C₁-C₄-haloalkyl, and C₀-C₄-alkylene-R^{1a}, wherein R^{1a} is independently selected from C₃-C₆ cycloalkyl, phenyl, and 4- to 6-membered heterocyclyl; wherein where R^{1a} is C₃-C₆ cycloalkyl or 4- to 6-membered heterocyclyl, R^{1a} is optionally substituted with from 1 to 4 R^{1b}, and where R^{1a} is phenyl, R^{1a} is optionally substituted with from 1 to 4 R^{1c};
   R² is independently selected from H, C₁-C₄-alkyl, C₁-C₄-haloalkyl, and C₀-C₄-alkylene-R^{2a}, wherein R^{2a} is independently selected from C₃-C₆ cycloalkyl, phenyl, and 4- to 6-membered heterocyclyl; wherein where R^{2a} is C₃-C₆ cycloalkyl or 4- to 6-membered heterocyclyl, R^{2a} is optionally substituted with from 1 to 4 R^{2b}, and where R^{2a} is phenyl, R^{2a} is optionally substituted with from 1 to 4 R^{2c};
R³ is independently selected from —C(O)OR^{3f}, —C(O)R^{3f}, —C(O)NR^{3g}R^{3f}, —S(O)₂OR^{3f}, —S(O)₂R^{3f}, —S(O)₂NR^{3g}R^{3f};
   R^{3f} is a linker covalently linked to an affinity tag or imaging probe;
   R^{3g} is independently selected from H, C₁-C₆-alkyl, and C₁-C₆-haloalkyl; R^{1b} and R^{2b} are each independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
   R^{1c} and R^{2c} are each independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
   R⁴ is independently at each occurrence selected from H and C₁-C₄-alkyl; or where two R⁴ groups are attached to the same nitrogen, those two R⁴ groups together with the nitrogen atom to which they are attached optionally form a 5- to 6-membered-heterocycloalkyl group optionally substituted with from 1 to 4 R⁵; and
   R⁵ are each independently at each occurrence selected from ═O, ═S, halo, nitro, cyano, C₁-C₄-alkyl, and C₁-C₄-haloalkyl.

Reactions in accordance with this aspect of the invention advantageously modify pseudouridine in what is substantially a single step and so permit a convenient and efficient modification of pseudouridine into a cyclised derivative, particularly when comprised within an RNA or any other molecule. The resulting cyclised derivative is linked to an affinity tag or imaging probe, thereby permitting the convenient isolation and/or identification of the derivative molecule using a variety of possible techniques, whether qualitative or quantitative in character.

The affinity tag may be one selected from the group comprising biotin, FLAG tag, His-tag, HA tag, Strep-tag, Avi-tag, GST, c-myc-tag, V5-tag, E-tag, S-tag, SBP-tag, poly (Glu)-tag, calmodulin tag.

The linker may be a flexible linker, a cleavable linker; optionally wherein the cleavable linker is a photocleavable linker. Particular linkers of the aforementioned types may be selected from (a) a polyethylene glycol (PEG); (b) a peptide; (c) a nucleic acid; or (d) an oligosaccharide.

Where a PEG linker is used, then the number of PEG units may be in the range n=1-12.

Where a peptide is used as the linker then it may have a molecular weight in the range of 100 to 5000 g/mol.

Where a nucleic acid is used as the linker, then it may have contain a number of nucleotides in the range 1 to 40 nucleotides.

Where an oligosaccharide is used as the linker, then it is preferably an oligosaccharide containing 1 to 40 monosaccharides.

When an imaging probe is used, then this may be selected from the group comprising fluorescent moieties, radionuclides and metal complexes.

Where the imaging probe is a fluorophore it may be selected from the group comprising a fluorescein, a rhodamine, BIODIPY, an Alexa fluor, a Cy dye or an ATTO dye. Examples of fluorescein fluorophores include FAM, HEX or VIC. Examples of rhodamine fluorophores include ROX, TAMRA, TEX 615. Examples of Alexa Fluor include Alexa Fluor 488, Alexa Fluor 532, Alexa Fluor 546, Alexa Fluor 594, Alexa Fluor 647, Alexa Fluor 660, Alexa Fluor 750. Examples of Cy Dye include Cy 3, Cy 5, Cy 5.5. Examples of ATTO Dye include ATTO 488, ATTO 532, ATTO 550, ATTO 565, ATTO Rho101, ATTO 590, ATTO 633, ATTO 647N. Particularly preferred fluorophores have an excitation maximum in the range of 350 to 850 nm; preferably ATT0488 or DY676.

In some instances, a fluorophore such as FAM can be used in conjunction with anti-FAM antibodies as a way of pulldown enrichment similarly to that described herein in relation to biotin-avidin.

Where pseudouridine is modified and the derivative includes a fluorophore, then for certain sample types the presence, location and optionally the amount or concentration of the fluorescent derivative can be determined in vitro. Suitable methods may include, for example, those described in Knutson, K. D., et al., (2018) Bioconjugate Chem. Vol 29(9): 2899-2903.

Where the imaging probe is a lanthanide complex it may be of Gd, Mn, Dy or Eu.

Where the imaging probe is a radionuclide complex it may be comprise ⁶⁴Cu, ⁶⁸Ga, ¹⁸F, ^{99m}Tc, ¹²³I, ¹²⁵I, ¹³¹I, ⁵⁷Co, ⁵¹Cr, ⁶⁷Ga, ⁶⁴Cu, ⁹⁰Y.

The invention also includes a method of isolating RNA comprising pseudouridine from a sample, comprising reacting the sample with a Michael Addition acceptor according to a method as hereinbefore defined, and wherein the pseudouridine is tagged with affinity tag, and then contacting the sample with a substrate comprising the binding partner to the affinity tag. The Michael Addition acceptor may already comprise an affinity tag as herein described, or the Michael Addition acceptor may comprise a reactive moiety to which an affinity tag may be reacted, whether before, after or simultaneously with the reaction with the pseudouridine. In this way, isolation of pseudouridine-containing RNA from a sample may be achieved. The substrate is preferably a solid phase substrate, for example agarose, cellulose, dextran, polyacrylamide, latex or controlled pore glass; and ideally the solid phase substrate is porous.

The affinity isolation of RNA using a tagged pseudouridine may be carried out as a batch or a column process, as will be familiar to a person of skill in the art. Generally, the series of steps involved comprise incubation of the sample with the substrate under conditions which permit the fullest possible binding of tagged RNA to the substrate. Then the unbound sample components are washed away from the substrate using suitable buffer or buffers, during which the tagged RNA remains bound to the substrate. Then the tagged RNA is eluted from the substrate using altered buffer conditions that dissociate the tagged RNA from the substrate. The tagged RNA can in this way be efficiently collected.

Advantageously, tagged RNA may be subjected to a pulldown enrichment process prior to further processing, e.g. involving sequencing or reverse transcription. This may be useful in connection with processing of RNA which has low abundance of pseudouridine sites.

In another aspect, solid phase affinity matrices can be used for the purpose of binding assays for the detection and optional quantitation of correspondingly affinity tagged pseudouridine present in a sample.

In a particularly preferred method of affinity isolation or quantitation of pseudouridine tagged RNA, the affinity tag is biotin and the binding partner of the solid phase is avidin or streptavidin. Alternatively, the affinity tag may be avidin or streptavidin and the binding partner of the solid phase is biotin.

In another aspect, the invention provides a method of visualising pseudouridine in a sample comprising RNA, comprising reacting the sample with a Michael Addition acceptor according to a method as hereinbefore defined, and wherein the pseudouridine is tagged with imaging probe, and subjecting the sample to a visualization procedure selected from optical observation, microscopical observation and image capture.

The samples in connection with methods of visualisation may comprise tissues or cells. In such samples where individual cells can be discriminated, this permits the location and frequency of pseudouridine to be observed and thereby correlated with cell type or cell location in the sample. Methods of visualisation of pseudouridine may also be combined with probes for particular RNA sequences of interest.

In another aspect, the invention provides a method of determining the presence and sequence location of pseudouridine comprised in a sample of RNA comprising:
(a) reacting at least a portion of the sample with a Michael Addition acceptor, wherein the Michael Addition acceptor is a compound according to Formula (I):

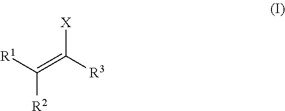

wherein:
X is independently selected from halo, tosyl, mesyl and triflyl;
   R¹ is independently selected from H, C₁-C₄-alkyl, C₁-C₄-haloalkyl, and C₀-C₄-alkylene-R^{1a}, wherein R^{1a} is independently selected from C₃-C₆ cycloalkyl, phenyl, and 4- to 6-membered heterocyclyl; wherein where R^{1a} is C₃-C₆ cycloalkyl or 4- to 6-membered heterocyclyl, R^{1a} is optionally substituted with from 1 to 4 R^{1b}, and where R^{1a} is phenyl, R^{1a} is optionally substituted with from 1 to 4 R^{1c};
   R² is independently selected from H, C₁-C₄-alkyl, C₁-C₄-haloalkyl, C₀-C₄-alkylene-R^{2a}, wherein R^{2a} is independently selected from C₃-C₆ cycloalkyl, phenyl, and 4- to 6-membered heterocyclyl; wherein where R^{2a} is C₃-C₆ cycloalkyl or 4- to 6-membered heterocyclyl, R^{2a} is optionally substituted with from 1 to 4 R^{2b}, and where R^{2a} is phenyl, R^{2a} is optionally substituted with from 1 to 4 R^{2c};
   R³ is independently selected from —C(O)OR^{3a}, —C(O)R^{3a}, —C(O)NR^{3b}R^{3b}, —CN, —NO₂, —S(O)₂OR^{3a}, —S(O)₂R^{3a}, —S(O)₂NR^{3b}R^{3b}, and 5- to 10-membered heteroaryl, wherein where R³ is 5- to 10-membered heteroaryl, R³ is optionally substituted with from 1 to 4 R^{3e};
   R^{3a} is independently selected from H, C₁-C₆-alkyl, C₁-C₆-haloalkyl, and C₀-C₆-alkylene-R^{3c};
   R^{3b} is independently selected from H, C₁-C₆-alkyl, C₁-C₆-haloalkyl, and C₀-C₆-alkylene-R^{3c}; or wherein two R^{3b} groups, together with the nitrogen atom to which they are attached form a 5- or 6-membered heterocycloalkyl group, optionally substituted with from 1 to 4 R^{3d};
   R^{3c} is independently selected from C₃-C₆ cycloalkyl, phenyl, and 4- to 6-membered heterocyclyl; wherein where R^{3c} is C₃-C₆ cycloalkyl or 4- to 6-membered heterocyclyl, R^{3c} is optionally substituted with from 1 to 4 R^{3d}, and where R^{3c} is phenyl, R^{3c} is optionally substituted with from 1 to 4 R^{3e};
   R^{1b}, R^{2b}, and R^{3d} are each independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
   R^{1c}, R^{2c}, and R^{3e} are each independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
   R⁴ is independently at each occurrence selected from H and C₁-C₄-alkyl; or where two R⁴ groups are attached to the same nitrogen, those two R⁴ groups together with the nitrogen atom to which they are attached optionally form a 5- to 6-membered-heterocycloalkyl group optionally substituted with from 1 to 4 R⁵; and
   R⁵ are each independently at each occurrence selected from ═O, ═S, halo, nitro, cyano, C₁-C₄-alkyl, and C₁-C₄-haloalkyl;
   (b) (i) sequencing the RNA; or
      (ii) reverse transcribing the RNA resulting from step (a) to provide cDNA and amplifying the cDNA;
   (c) sequencing the DNA of step (b)(ii);
   (d) comparing the DNA sequence of step (c) with a reference DNA sequence to identify the sequence positions of guanine (G) in the sequence which are adenine (A) in the reference sequence, the position of G in the DNA sequence being the positions of a pseudouridine in the corresponding RNA sequence.

The reference sequence may be obtained from a separate portion of the RNA sample which is not subjected to Michael Addition reaction of step (a), but which is sequenced according to step (b)(i); or reverse transcribed according to step (b)(ii) and sequenced according to step (c).

The amplification of cDNA may employ an isothermal method of amplification; wherein the isothermal method of amplification is selected from polymerase chain reaction (PCR) strand-displacement amplification (SDA), rolling-circle amplification (RCA), whole-genome amplification (WGA), loop-mediated isothermal amplification (LAMP), helicase-dependent amplification (HDA), and multiple displacement amplification (MDA); optionally wherein a one-step RT-PCT is used.

The reverse transcription of step (b) may use a reverse transcriptase enzyme (i.e. “RNA-directed DNA polymerase”; EC 2.7.7.49); optionally a reverse transcriptase enzyme selected from: Maxima H-, SuperScript II, SuperScript III, SuperScript IV, ProtoScript II, SMARTScribe, PrimeScript II, HiScript III, MMLV, AMV, TGIRT-III or recombinant HIV; preferably Maxima H-, SuperScript IV and TGIRT-III; more preferably Maxima H-. Also Marathon reverse transcriptase or Induro reverse transcriptase may be used, and particularly in methods where tRNA is involved because like TGIRT-III, these are group II intron-encoded RT enzymes.

In methods of the invention wherein the modified RNA is sequenced directly, a preferred method of sequence is ion torrent sequencing, for example by using the system of Oxford Nanopore. In this direct sequencing of RNA, modified pseudouridine residues may detected directly. Optionally a control or reference sample of the RNA may be used which is not subjected to pseudouridine modification and which is also sequenced using the ion torrent sequencing method, allowing for a comparative side by side sequencing of modified and unmodified RNA.

Where a method of the invention includes a sequencing method, any suitable method of sequencing familiar to a person of skill in the art may be used. Examples of such sequencing methods are explained briefly below.

In single-molecule real-time (SMRT) sequencing (Pacific Biosciences) the DNA is synthesized in zero-mode wave-guides (ZMWs). These are wells with capture sequences and include an unmodified polymerase and fluorescently labelled nucleotides in solution. Only fluorescence taking place in the bottom of the well is detected. SMRT sequencing allows reads of 20,000 nucleotides or more, with average read lengths of 5 kilobases.

Ion Torrent sequencing (Thermo Fisher Scientific) uses normal sequencing chemistry with a special semiconductor device. The basis of the technique relies on detecting by their charge, hydrogen ions released during polymerization of DNA. A microwell with the template DNA strand to be sequenced is exposed to just one type of nucleotide at a time. If the nucleotide is complementary to the template at the position being synthesized than it is incorporated and releases a hydrogen ion which is measured to confirm that a reaction (of that particular base) has occurred. This sequencing method can provide individual read lengths of about 800 bp. As noted above, another version of this sequencing is Oxford Nanopore and this is useful in embodiments of the methods of the invention which require direct sequencing of RNA molecules modified in accordance with methods of the invention.

Pyrosequencing (454) (Roche Diagnostics) uses water droplets in an oil solution (emulsion PCR) as the environment for DNA synthesis. Each droplet contains a single DNA template attached to a single primer-coated bead that forms a clonal colony. The sequencing machine has picoliter-volume wells, each with a single bead and sequencing enzymes. Luciferase is used to generate light for detecting the incorporation event of individual nucleotides attaching to the nascent DNA strand. This method provides read lengths of about 700 bp.

Sequencing by synthesis (Illumina) is available in a number of versions, such as MiniSeq, NextSeq, MiSeq, HiSeq 2500, HiSeq 3/4000 and HiSeq X. In this method, DNA molecules and primers are first attached on a slide or flow cell and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labelled nucleotides. Then the dye, along with the terminal 3′ blocker, is chemically removed from the DNA, allowing for the next cycle to begin. The methods can provide read lengths in the range 50-600 bp.

Sequencing by ligation (SOLiD sequencing) (Thermo Fisher) employs sequencing by ligation. A pool of all possible oligonucleotides of a fixed length are labelled according to the sequenced position. Oligonucleotides are annealed and ligated; the preferential ligation by DNA ligase for matching sequences results in a signal informative of the nucleotide at that position. Prior to sequencing DNA is amplified by emulsion PCR and the resulting beads, each containing single copies of the same DNA molecule, are deposited on a glass slide. Reads of 50 bp are obtained.

Other methods of sequencing will be apparent to a person of skill in the art and readily incorporated into the methods of the invention.

Preferably, in each aspect of the invention, X is selected from Br, Cl, and I.

Also, preferably, in each aspect of the invention, at least one of R¹ and R² is H; optionally wherein both R¹ and R² are H.

R³ is preferably independently selected from —C(O)OR^{3a}, —C(O)R^{3a}, —C(O)NR^{3b}R^{3b}, and —CN; more preferably wherein R³ is —C(O)NR^{3b}R^{3b}, optionally wherein R³ is —C(O)NH₂.

In each aspect of the invention, the compound of Formula (I) is preferably selected from:

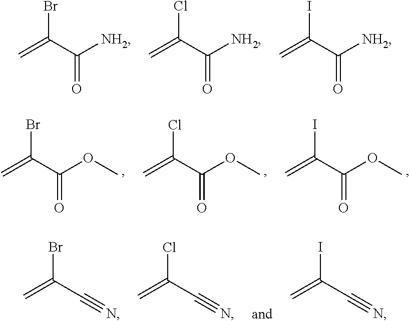

Suitable reaction conditions are used in the methods of the invention, for example, the Michael Addition reaction is performed at a pH in the range of from about 7.0 to about 9.5.

The reaction of an RNA molecule with the Michael Addition acceptor may be performed at a pH in the range about pH 7.0 to about pH 9.5. The term “in the range” herein includes the upper and lower pH limits of the range. The pH may instead be in any of the following ranges: about pH 7.1 to about pH 9.5, about pH 7.2 to about pH 9.5, about pH 7.3 to about pH 9.5, about pH 7.4 to about pH 9.5, about pH 7.5 to about pH 9.5, about pH 7.6 to about pH 9.5, about pH 7.7 to about pH 9.5, about pH 7.8 to about pH 9.5, about pH 7.9 to about pH 9.5, about pH 8.0 to about pH 9.5, about pH 8.1 to about pH 9.5, about pH 8.2 to about pH 9.5, about pH 8.3 to about pH 9.5, about pH 8.4 to about pH 9.5, or about pH 8.5 to about pH 9.5. Or the pH may be in any of the following ranges: about pH 7.0 to about pH 9.4, about pH 7.0 to about pH 9.3, about pH 7.0 to about pH 9.2, about pH 7.0 to about pH 9.1, about pH 7.0 to about pH 9.0, about pH 7.0 to about pH 8.9, about pH 7.0 to about pH 8.8, about pH 7.0 to about pH 8.7, about pH 7.0 to about pH 8.6, or about pH 7.0 to about pH 8.5. Or the pH may be in any of the following ranges: about pH 7.5 to about pH 9.4, about pH 7.5 to about pH 9.3, about pH 7.5 to about pH 9.2, about pH 7.5 to about pH 9.1, about pH 7.5 to about pH 9.0, about pH 7.5 to about pH 8.9, about pH 7.5 to about pH 8.8, about pH 7.5 to about pH 8.7, about pH 7.5 to about pH 8.6, or about pH 7.5 to about pH 8.5. Or the pH may be in any of the following ranges: about pH 8.0 to about pH 9.4, about pH 8.0 to about pH 9.3, about pH 8.0 to about pH 9.2, about pH 8.0 to about pH 9.1, about pH 8.0 to about pH 9.0, about pH 8.0 to about pH 8.9, about pH 8.0 to about pH 8.8, about pH 8.0 to about pH 8.7, about pH 8.0 to about pH 8.6, or about pH 8.0 to about pH 8.5. Or the pH may be in any of the following ranges: about pH 7.1 to about pH 9.0, about pH 7.2 to about pH 9.0, about pH 7.3 to about pH 9.0, about pH 7.4 to about pH 9.0, about pH 7.5 to about pH 9.0, about pH 7.6 to about pH 9.0, about pH 7.7 to about pH 9.0, about pH 7.8 to about pH 9.0, about pH 7.9 to about pH 9.0, about pH 8.0 to about pH 9.0, about pH 8.1 to about pH 9.0, about pH 8.2 to about pH 9.0, about pH 8.3 to about pH 9.0, about pH 8.4 to about pH 9.0, or about pH 8.5 to about pH 9.0.

The reaction may preferably be performed at a pH selected from any of: about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, or about pH 9.5.

Suitable concentrations of reagents are used in the methods of the invention, for example, wherein the Michael Addition acceptor is present at a concentration in the range from about 10 mM to about 2M; preferably in the range from about 100 mM to about 500 mM; more preferably about 250 mM.

Also, suitable reaction conditions of temperature are used, ideally at ambient pressures, wherein the Michael Addition reaction is performed at a temperature in the range of from about 25° C. to about 95° C.; preferably from about 65° C. to about 95° C.; more preferably about 85° C. The reaction of an RNA molecule with the Michael Addition acceptor may be performed at a temperature in the range of about 40° C. to about 85° C. As with pH, the term “in the range” herein includes the upper and lower temperature limits of the range. For the reaction in accordance with the invention, the upper threshold temperature may selected from about 84° C., about 83° C., about 82° C., about 81° C., about 80° C., about 79° C., about 78° C., about 77° C. or about 76° C. This may be combined with a lower threshold temperate of about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., or about 74° C. The reaction in accordance with the invention may be carried out at any of the following temperatures selected from: about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., or about 74° C., about 75° C., about 76° C., about 77° C., about 78° C., about 79° C., about 80° C., about 81° C., about 82° C., about 83° C., about 84° C., or about 85° C.

Additionally, suitable reaction times are used dependent on temperature, for example wherein the Michael Addition reaction is performed at a temperature of about 70° C. or greater for a time in the range of from about 5 minutes to about 2 hours; or at a temperature of about 70° C. or less for a time in the range of from about 2 hours to about 16 hours.

The Michael addition reaction in accordance with the invention may be performed at a temperature of about 70° C. or greater for a period of time in the range 10 minutes to 60 minutes. As with pH and temperature, the term “in the range” herein includes the upper and lower limits of time for the specified ranges. For the reaction in accordance with the invention, the upper threshold time may selected from about 60 minutes, about 59 minutes, about 58 minutes, about 57 minutes, about 56 minutes, about 55 minutes, about 54 minutes, about 53 minutes, about 52 minutes, about 51 minutes, about 50 minutes, about 49 minutes, about 48 minutes, about 47 minutes, about 46 minutes, about 45 minutes, about 44 minutes, about 43 minutes, about 42 minutes, about 41 minutes, about 40 minutes, about 39 minutes, about 38 minutes, about 37 minutes, about 36 minutes, about 35 minutes. The aforementioned may be combined with a lower threshold time limit of about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, or about 30 minutes.

The Michael addition reaction in accordance with the invention may be performed for a period of time selected from any of: about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or about 60 minutes. The invention does not exclude the possibility of reaction time in excess of 60 minutes, e.g. about 70 minutes, about 80 minutes, about 90 minutes or about 100 minutes.

In any of the aspects of the invention, the RNA molecule may be selected from one or more of mRNA, tRNA, rRNA, snRNA, miRNA, lncRNA or circRNA. Particularly, the RNA molecule may be derived from a biological sample.

The invention also includes compositions comprising a Michael Addition acceptor according to Formula (I) as hereinbefore defined, and an RNA molecule.

The invention also includes RNA molecules comprising at least one carbamido-1, O2-ethano ψ, nce1,2ψ. Such modified RNA molecules are a reaction product of an RNA molecule comprising one or more ψ residues. Reaction of the RNA molecule comprising one or more ψ residues results in an RNA molecule wherein at least one of said ψ residues is modified to carbamido-1, O2-ethano ψ, nce1,2ψ.

In some aspects, a proportion of the ψ residues are modified, and this proportion may be any in the range 1% to 100% of all ψ residues in the RNA molecule. The proportion may be selected from any of at least 1%, at least 2%, at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 96%, at least 97%, at least 98% or at least 99%.

The invention includes compositions comprising a modified RNA molecule as herein defined.

The methods of the invention may be used to determine the presence and sequence location of pseudouridine in any RNA molecule. The RNA molecule may be selected from any of an mRNA molecule, a tRNA molecule, an rRNA molecule, an snRNA molecule, an miRNA molecule, or an lncRNA molecule. The RNA molecule may be from a cfRNA sample. Also, the RNA molecule may be an RNA molecule of a plurality of RNA molecules, wherein the methods of the invention further comprises quantifying the number of pseudouridines in the plurality of RNA molecules.

The RNA used in methods of the invention may be obtained from a sample, more particularly an environmental or biological sample. A biological sample includes a sample taken from any organism. Included are medical or veterinary samples and the skilled person is well aware of the range of possible ways of obtaining such samples. For example, methods of biopsy performed on the body, such as fine needle aspiration, core needle biopsy, vacuum assisted biopsy, incisional biopsy, excisional biopsy, punch biopsy, shave biopsy or skin biopsy. Normal tissue and corresponding tumorous or cancerous tissue may be sampled and compared. Pseudouridine residues in RNA may be compared as between normal and cancerous samples in the study of development of cancer and metastasis. Other samples from which RNA may be obtained include blood, sweat, hair follicle, buccal tissue, tears, menses, faeces, or saliva.

Particularly preferred samples include those of blood, cell free RNA and liquid biopsy.

A sample may include but is not limited to, tissue, cells, or biological material from cells or derived from cells. Such cells may be selected from any of bone marrow mononuclear cells, buffy coat, dissociated tumour cells, epithelial cells, fibroblasts, hepatocytes, mesenchymal stem cells, myoblasts, PBMCS, purified immune cells (e.g. T Cell, B Cells, NK cells etc.) or red blood cells (RBCs).

The biological sample may be a homogeneous or heterogeneous population of cells or tissues. In some instances, the sample can be free of cells and so for example serum or plasma.

Biofluids may provide a suitable sample for RNA for examination, for example blood, bile, bone marrow aspirate, breast milk, plasma, saliva, cerebral spinal fluid (CSF), serum, stool, sputum, oral or nasal fluids, urine or synovial fluid.

Tissues may provide suitable samples comprising RNA for examination in accordance with methods of the invention, and such tissue may have been collected through biopsy or surgical procedure. Some tissues may also have been fixed, frozen or processed for analysis.

Cell free nucleic acid samples may be interrogated in accordance with the methods of the invention. Such samples comprise cell-free DNA (cfDNA) and/or cell-free RNA (cfRNA). The nucleic acid in such samples may be isolated and optionally purified to varying degree from the sample.

RNA may be extracted from samples prior to reaction with a Michael Addition Acceptor in accordance with the invention. Commercial RNA extraction kits may be used and particular examples of such kits include those made and sold by (a) New England Biolabs Monarch® RNA MiniPrep kit, (b) Qiagen AllPrep® PowerViral® DNA/RNA kit, (c) Zymo Quick RNA™-Viral Fecal/Soil Microbe Microprep kit, or (d) Zymo Quick RNA™-Viral with Inhibitor Removal. Other suitable commercial kits may be used including Quick-RNA Miniprep Kit (Zymo) or the Invitrogen™ TRIzol™ Plus RNA Purification Kit.

Suitable samples to which the present invention is usefully applied may comprise at least, at most, or about 1 ng, 2 ng, 3 ng, 4 ng, 5 ng, 6 ng, 7 ng, 8 ng, 9 ng or 10 ng of nucleic acid. Also, about 20 ng, about 30 ng, 40 ng, 50 ng, 60 ng, 70 ng, 80 ng, 90 ng, 100 ng, 200 ng, 300 ng, 400 ng, 500 ng, 600 ng, 700 ng, 800 ng, 900 ng, 1 μg, 2 μg, 3 μg, 4 μg, 5 μg, 6 μg, 7 μg, 8 μg, 9 μg or 10 μg. The sample may comprise an amount of nucleic acid within a range of nucleic acid weight defined by any one of the above as the lower limit and any other of the above as the upper limit.

Methods of the invention have utility in relation to clinical and biological research and diagnostics. The kinds of RNA molecules which can be analysed using methods of the invention may include messenger RNA (mRNA), transfer RNA (tRNA), ribosomal RNA (rRNA), long noncoding RNA (lncRNA), short noncoding RNA (sncRNA), microRNA (miRNA), small nuclear RNA (snRNA), small nucleolar RNA (snoRNA), small interfering RNA (siRNA), short hairpin RNA (shRNA) and circular RNA (circRNA). The evaluation is for the determination of pseudouridine residues in the sequences of these RNA molecules.

The presence of pseudouridine residues may serve as novel biomarkers for a disease or condition. Methods of the invention can therefore be applied in discovering such biomarkers amongst patient samples. Similarly, such biomarkers can be used to monitor for disease progression and/or for patient responsiveness to drugs or other treatments. The method of the invention may be of particular usefulness in the science of oncology and the study of cancer. Examples of cancers which are amendable to study using methods of the invention may include the commoner types of cancer such as bladder cancer, breast cancer, colon cancer, rectal cancer, endometrial cancer, kidney cancer, leukaemia, liver cancer, lung cancer, melanoma, non-Hodgkin lymphoma, pancreatic cancer, prostate cancer and thyroid cancer. Other, less common cancers of any type may be studied or monitored in accordance with methods of the invention.

There is growing knowledge about the relationship between pseudouridine in tRNA and cancer. For example, in relation to glioblastoma (Cui, Q. et al., (2021) “Targeting PUS7 suppresses tRNA pseudouridylation and glioblastoma tumorigenesis” Nat Cancer 2(9): 932-949) and acute myeloid leukaemia (Guzzi, N. et al'., (2022) “Pseudouridine-modified tRNA fragments repress aberrant protein synthesis and predict leukemic progression in myelodysplastic syndrome” Nat. Cell Biol. 24: 299-306). Method of the invention are expected to further the knowledge in these and other cancer types.

The invention also provides a kit for modifying a pseudouridine comprising: (a) a solution comprising a Michael Addition acceptor as hereinbefore defined; (b) instructions for reacting a sample comprising pseudouridine with the solution.

The invention further comprises a kit for tagging or labelling pseudouridine comprised in RNA, comprising: (a) a solution comprising a Michael Addition acceptor as hereinbefore defined; (b) instructions for reacting an RNA with the solution.

The invention further comprises a kit for determining the presence of sequence location of pseudouridine comprised in RNA, comprising: (a) a solution comprising a Michael Addition acceptor as hereinbefore defined; (b) instructions for reacting an RNA with the solution.

In any of the aforementioned, a kit of the invention may comprise one or more of: (a) one or more buffers, (b) a reverse transcriptase enzyme, or (c) a DNA polymerase enzyme. The kit may also include one or more containers wherein each container contains one of the elements of the kit.

Where there is a reverse transcriptase enzyme this may be selected from: Maxima H-, SuperScript II, SuperScript III, SuperScript IV, ProtoScript II, SMARTScribe, PrimeScript II, HiScript III, MMLV, AMV, TGIRT-III, recombinant HIV, Marathon reverse transcriptase or Induro reverse transcriptase; preferably Maxima H-, SuperScript IV and TGIRT-III; more preferably Maxima H-.

Where there is a DNA polymerase this may be selected from Taq DNA polymerase, Bst DNA polymerase or Bsu DNA polymerase.

A kit in accordance with the invention may comprise instructions for the use thereof. The instructions may be for how to incubate a nucleic acid molecule sample with an included solution, e.g. the Michael Addition acceptor as defined herein. The instruction may state the conditions necessary for modifying at least a portion of pseudouridines in the nucleic acid molecule. Such conditions may include, for example, pH conditions, temperature conditions, incubation time, as described elsewhere herein. Examples of such conditions necessary for modification of pseudouridines are disclosed herein. The instructions may include statements about incubating the sample with the Michael Addition acceptor for a given period of time, as disclosed herein.

In further aspects of the invention, the kit may comprise a polynucleotide kinase enzyme, such as T4 polynucleotide kinase.

A kit may optionally provide additional components that are useful in the procedure. These optional components include buffers, capture reagents, developing reagents, labels, reacting surfaces, means for detection or control samples. As well as instructions, kits may include interpretive information.

Certain kits may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 750, 1,000 or more probes, primers or primer sets, synthetic molecules or inhibitors

Kits may comprise components which may be individually packaged or placed in a container, such as a tube, bottle, vial, syringe, or other kind of container. Individual components may be present in a kit as a concentrate and diluted prior to use.

Some kits may contain control nucleic acids, for example an RNA molecule that does not contain pseudouridine as a negative control, and an RNA molecule that contains pseudouridine as a positive control.

In an embodiment, X is halo. In an embodiment, X is selected from Cl (chloro), Br (bromo), and I (iodo). In an embodiment, X is Br.

In an embodiment, R¹ is independently selected from H, C₁-C₄-alkyl, and C₁-C₄-haloalkyl. In an embodiment, R¹ is independently selected from H, C₁-C₂-alkyl, and C₁-C₂-haloalkyl. In an embodiment, R¹ is H.

In an embodiment, R² is independently selected from H, C₁-C₄-alkyl, and C₁-C₄-haloalkyl. In an embodiment, R² is independently selected from H, C₁-C₂-alkyl, and C₁-C₂-haloalkyl. In an embodiment, R² is H.

In an embodiment, at least one of R¹ and R² is H. In an embodiment, R¹ and R² are both H.

In an embodiment, R³ is independently selected from —C(O)OR^{3a}, —C(O)R^{3a}, and —C(O)NR^{3b}R^{3b}. In an embodiment, R³ is independently selected from —CN, and —NO₂. In an embodiment R³ is independently selected from —S(O)₂OR^{3a}, —S(O)₂R^{3a}, and —S(O)₂NR^{3b}R^{3b}.

In an embodiment, R³ is 5 to 10-membered heteroaryl.

In an embodiment, R³ is —C(O)OR^{3a}. In an embodiment R³ is —C(O)R^{3a}. In an embodiment R³ is —C(O)NR^{3b}R^{3b}.

In an embodiment, R^{3a} is H. In an embodiment R^{3a} is C₁-C₆-alkyl. In an embodiment R^{3a} is C₁-C₆-haloalkyl. In an embodiment R^{3a} is C₀-C₆-alkylene-R^{3c}.

In an embodiment, R^{3b} is H. In an embodiment R^{3b} is C₁-C₆-alkyl. In an embodiment R^{3b} is C₁-C₆-haloalkyl. In an embodiment R^{3b} is C₀-C₆-alkylene-R^{3c}.

In an embodiment where R³ is —C(O)NR^{3b}R^{3b}, one R^{3b} is H and the other R^{3b} is as defined herein.

In an embodiment, R^{3c} is C₃-C₆ cycloalkyl. In an embodiment, R^{3c} is phenyl. In an embodiment, R^{3c} is 4- to 6-membered heterocyclyl.

In an embodiment, R³ is —C(O)NH₂. In an embodiment, R³ is —C(O)OMe. In an embodiment, R³ is —CN.

In an embodiment R^{1b}, R^{2b}, and R^{3d} are each independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl.

In an embodiment R^{1b}, R^{2b}, and R^{3d} are each independently at each occurrence selected from ═O, ═S, halo, nitro, and cyano.

In an embodiment, R^{1c}, R^{2c}, and R^{3e} are each independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl.

In an embodiment, R^{1c}, R^{2c}, and R^{3e} are each independently at each occurrence selected from halo, nitro, and cyano.

In an embodiment, R⁴ is H. In an embodiment, R⁴ is C₁-C₄-alkyl.

In embodiments relating to the method of tagging or labelling pseudouridine in a sample, R³ is independently selected from —C(O)OR^{3f}, —C(O)R³¹, and —C(O)NR^{3g}R^{3f}. In embodiments relating to the method of tagging or labelling pseudouridine in a sample, R³ is —C(O)NR^{3g}R^{3f}.

In embodiments, R^{3g} is H. In embodiments, R^{3g} is C₁-C₆-alkyl, optionally C₁-C₃-alkyl. In embodiments, R^{3g} is C₁-C₆-haloalkyl, optionally C₁-C₃-haloalkyl.

In embodiments, R^{3f} is a linker covalently linked to an affinity tag. In embodiments, R^{3f} is a linker covalently linked to an imaging probe.

In embodiments, the affinity tag is selected from the group comprising biotin, FLAG tag, His-tag, HA tag, Strep-tag, Avi-tag, GST, c-myc-tag, V5-tag, E-tag, S-tag, SBP-tag, poly (Glu)-tag, calmodulin tag.

In embodiments, the affinity tag is biotin.

In embodiments, the imaging probe is selected from the group comprising fluorescent moieties, radionuclides and metal complexes.

In embodiments, the imaging probe is selected from the group comprising (a) fluorophores with an excitation maximum in the range of 350 to 850 nm; preferably ATT0488 or DY676; (b) lanthanide complexes; preferably of Gd, Mn, Dy, Eu; (c) radionuclide complexes ⁶⁴Cu, ⁶⁸Ga, ¹⁸F, ^{99m}Tc, ¹²³I, ¹²⁵I, ¹³¹I, ⁵⁷Co, ⁵¹Cr, ⁶⁷Ga, ⁶⁴Cu, ⁹⁰Y.

In embodiments, the imaging probe is selected from the group comprising Fluorescein (FAM, HEX, VIC), Rhodamine (ROX, TAMRA, TEX 615), BODIPY, Alexa Fluor (Alexa Fluor 488, Alexa Fluor 532, Alexa Fluor 546, Alexa Fluor 594, Alexa Fluor 647, Alexa Fluor 660, Alexa Fluor 750), Cy Dye (Cy 3, Cy 5, Cy 5.5), and ATTO Dye (ATTO 488, ATTO 532, ATTO 550, ATTO 565, ATTO Rho101, ATTO 590, ATTO 633, ATTO 647N).

In an embodiment, the linker is a flexible linker; optionally selected from the group comprising (a) a polyethylene glycol, (b) a peptide; preferably a peptide having a molecular weight in the range of 100 to 5000 g/mol, (c) a nucleic acid; preferably a nucleic acid containing 1 to 40 nucleotides, or (d) an oligosaccharide; preferably an oligosaccharide containing 1 to 40 monosaccharides.

In an embodiment, R^{3f} may have the structure:

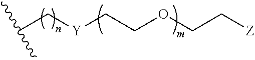

wherein:
n is an integer selected from 1, 2, 3, 4, 5, and 6; m is an integer selected from 1, 2, 3, 4, 5, 6,7,8,9,10, 11, and 12;
Z is an affinity tag or imaging probe as defined herein; and
ψ is a diazao (—N═N—) group, a disulphide (—S—S—) group, a Dde group, or a photocleavable group.

### BRIEF DESCRIPTION OF THE DRAWINGS

Embodiments of the invention are further described hereinafter with reference to the accompanying drawings, in which:

FIG. 1 is a FIG. 1. Schematic overview of BACS reaction. After BACS labelling, O² of nce^{1,2}ψ could not serve as a hydrogen bond acceptor and therefore would be read as C during RT.

FIG. 2a. MALDI characterization of BACS labelling of modified (Y) 10mer product and unmodified (U) 10 mer.

FIG. 2b shows consumption rates of ψ in HeLa total RNA and 1.8-kb 10% ψ-modified RNA upon BACS treatment, quantified by UHPLC-MS/MS. Data are presented as means of two independent experiments.

FIG. 3 shows mutation ratios of ψ sites in 72mer model RNA after BACS treatment. Data are shown as means±s.d. of ten independent experiments (n=10).

FIG. 4 shows cumulative (upper bar chart) and motif-dependent (lower tabulations) results of BACS conversion rates and false-positive rates on synthetic 30mer NNYNN and NNUNN spike-in. Data are shown as means±s.d. of three and eight independent experiments for NNψNN (n=3) and NNUNN (n=8) spike-in, respectively.

FIG. 5a shows BACS calibration curve for quantification of ψ stoichiometry in NNUNN motif. Experiment was performed once. FIG. 5b shows BACS calibration curve for quantification of ψ stoichiometry in UGUAG motif. Experiment was performed once.

FIG. 6 is a flowchart of BACS library construction.

FIG. 7 is chart showing the numbers of ψ sites identified in human cy-rRNAs and mt-rRNAs.

FIG. 8a is a chart showing the rates of BACS (darker) and control (lighter) samples in 28S rRNA. FIG. 8b is a chart showing the rates of BACS (darker) and control (lighter) samples in 18S rRNA. FIG. 8c is a chart showing the rates of BACS (darker) and control (lighter) samples in 5.8S rRNA.

FIG. 9a shows BACS conversion rates of ψ sites identified in 18S rRNA—data are shown as means±s.d. of four independent experiments (n=4). FIG. 9b shows modification levels of ψ sites detected in 18S rRNA—data are shown as means±s.d. of four independent experiments (n=4). FIG. 9c shows BACS conversion rates of ψ sites identified in 28S rRNA—data are shown as means±s.d. of four independent experiments (n=4). FIG. 9d shows modification levels of ψ sites detected in 28S rRNA—data are shown as means±s.d. of four independent experiments (n=4).

FIG. 10 shows a correlation density plot between two biological replicates of BACS. The degree of shading represents density.

FIG. 11 is a venn diagram illustrating the overlap of ψ sites detected in human cy-rRNAs between BACS and SILNAS MS.

FIG. 12 is comparison of the conversion rates in cy-rRNAs between BACS and control samples.

FIG. 13 is a comparison of the conversion rates of BACS with the deletion rates of BID-seq and PRAISE for selected ψ sites in 18S rRNA. Because BID-seq and PRAISE cannot quantify multiple ψ sites (2) located in the same consecutive uridine context, these sites were excluded.

FIG. 14 is a comparison of the conversion rates of BACS with the deletion rates of BID-seq and PRAISE for selected ψ sites in 28S rRNA. Because BID-seq and PRAISE cannot quantify multiple ψ sites (2) located in the same consecutive uridine context, these sites were excluded.

FIG. 15 shows a comparison of the modification levels of ψ sites in cy-rRNAs and mt-rRNAs. Boxplots indicate medians, quantiles, extreme values, and outliers.

FIG. 16 shows how BACS can detect ψ sites adjacent to one or more U and densely modified ψ sites. Blue and red color denote C and T bases, respectively. After BACS, ψ sites were identified by U-to-C mutation.

FIG. 17 shows how BACS results are not be influenced by other modified uridine bases. Blue and red color denote C and T bases, respectively. By comparing the BACS results with untreated control, m1acp3ψ and m3U sites can be easily filtered out.

FIG. 18 shows numbers of ψ sites identified in human splicesomal snRNAs.

FIG. 19 is a venn diagram illustrating the overlap of ψ sites detected in human splicesomal snRNAs between BACS and SILNAS MS.

FIG. 20 shows the conversion rates of BACS (darker) and control (lighter) samples in U2 snRNA. Data are presented as means of four independent experiments.

FIG. 21 shows conversion rates of BACS (darker) and control (lighter) samples in U4atac snRNA, showing the novel ψ₁₁ and known ψ₁₂ site. Data are presented as means of four independent experiments.

FIG. 22 shows base pairing interactions between U4atac and U6atac snRNAs in stem II region. The novel ψ₁₁ site is on the right of the known ψ₁₂ site.

FIG. 23 shows numbers of ψ sites identified in human snoRNAs and TERC.

FIG. 24a is a venn diagram illustrating the overlap of ψ sites detected in human snoRNAs between BACS and Y-seq. FIG. 24b is a venn diagram illustrating the overlap of ψ sites detected in human snoRNAs between BACS and BID-seq.

FIG. 25 shows the numbers of ψ sites with high (50-100%, upper), medium (20-50%, middle), and low (5-20%, lower) modification levels identified in box C/D snoRNAs, box H/ACA snoRNAs, and scaRNAs.

FIG. 26 shows modification level distributions of ψ sites in box C/D snoRNAs, box H/ACA snoRNAs, and scaRNAs. Boxplots visualize all ψ sites in each class of snoRNAs, indicating medians, quantiles, and extreme values.

FIG. 27 shows the metagene profile of ψ sites in box C/D snoRNAs. Respective shading denotes the boxes C/C′ and D/D′

FIG. 28 shows the metagene profile of ψ sites in box H/ACA snoRNAs. Respective shading denotes the boxes H and ACA.

FIG. 29 shows potential base pairing interactions between snoRNAs (darker) and their targets in rRNA (lighter) for box C/D snoRNAs. Identified snoRNA 4) sites are highlighted with position number. 2′-O-methylation targets in rRNA are underlined.

FIG. 30 shows potential base pairing interactions between snoRNAs (darker) and their targets in rRNA (lighter) for box H/ACA snoRNAs. Identified snoRNA ψ sites are highlighted with position number. 2′-O-methylation targets in rRNA are underlined.

FIG. 31 shows ψ modification levels in TERC, with each identified ψ site labeled accordingly. Data are presented as means of four independent experiments.

FIG. 32 shows the distributions of ψ sites identified in each human cy-tRNA isoacceptor family (left) and mt-tRNA (right).

FIG. 33 shows medians of ψ sites identified per tRNA in each human cy-tRNA (left) and mt-tRNA (right) isoacceptor family. (n/a=not applicable).

FIG. 34 is a heatmap showing the modification levels of high-confidence ψ sites in human cy-tRNAs. Only one representative tRNA isodecoder was presented for each isoacceptor family.

FIG. 35 shows an integrated view of the ψ profiles of human cy-tRNA (left) and mt-tRNA (right).

FIG. 36 is a comparison of the modification levels of ψ sites at selected positions of human cy-tRNAs. Boxplots visualize all ψ sites at each position, indicating medians, quantiles, and extreme values.

FIG. 37 is a venn diagram illustrating the overlap of ψ sites in human mt-RNAs reported by BACS and a previously published dataset.

FIG. 38 is a heatmap of the ψ modification levels in human mt-tRNAs.

FIG. 39 is comparison of the modification levels of ψ sites at selected positions of human mt-tRNAs. Boxplots visualize all ψ sites at each position, indicating medians, quantiles, and extreme values.

FIG. 40 is a schematic overview of Dimroth rearrangement of m¹A to m⁶A.

FIG. 41 is a comparison of the mutation rates of known m¹A sites in control (upper) and BACS (lower) samples. Boxplots indicate medians, quantiles, and extreme values.

FIG. 42 is a heatmap showing the changes in mutation rates of all adenosine sites in human mt-tRNAs upon BACS treatment. Degree of shading indicates an increase or decrease of mutation rates.

FIG. 43 shows the distribution of mapped reads in control libraries for polyA-tailed RNA. Data are representative of two independent experiments.

FIG. 44 shows the numbers of 4) sites with high (50-100%, upper), medium (20-50%, middle), and low (5-20%, lower) modification levels identified in HeLa polyA-tailed RNA.

FIG. 45 shows the modification level distribution of ψ sites in HeLa polyA-tailed RNA.

FIG. 46 shows an example of genome browser view presenting a highly modified ψ site. Upper shading denotes T counts. Lower shading denotes C counts.

FIG. 47 shows a comparison of the ψ modification levels in different RNA species. Boxplots indicate medians, quantiles, and extreme values.

FIG. 48 shows the distribution of ψ sites within different features of HeLa mRNA and ncRNA.

FIG. 49 shows the metagene profile of ψ sites in HeLa mRNA.

FIG. 50 shows gene ontology enrichment analysis (biological process) for HeLa mRNA ψ sites.

FIG. 51 shows a correlation density plot of RNA expression levels between BACS and control samples. The degree of shading represents density. Data are representative of two independent experiments.

FIG. 52 shows a correlation of HeLa RNA expression levels between BACS and BID-seq input libraries. Pearson's r values are shown.

FIG. 53 shows the distribution of mRNA ψ sites within single and consecutive uridine contexts.

FIG. 54 shows the motif frequency of ψ sites in HeLa mRNA.

FIG. 55 shows modification level distributions of HeLa mRNA ψ sites within selected motifs, with medians indicated in each plot.

FIG. 56 shows a comparison of the ψ modification levels in selected motifs between tRNA (upper) and mRNA (lower). Boxplots indicate medians, quantiles, and extreme values.

FIG. 57 shows the numbers of mRNA ψ sites located in different codons.

FIG. 58 shows the codons encoding different amino acids,

FIG. 59 shows the numbers of mRNA ψ sites located in different codon positions.

FIG. 60 shows a venn diagram illustrating the overlap of mRNA ψ sites between BACS and the “highest confidence” list in a consolidated CMC-based dataset.

FIG. 61 shows a venn diagram illustrating the overlap of mRNA ψ sites between BACS and the “high confidence” list in a consolidated CMC-based dataset.

FIG. 62 shows a venn diagram illustrating the overlap of mRNA ψ sites between BACS and the “high confidence” list in a consolidated CMC-based dataset. Only ψ sites consistently detected across more than 8 samples in the “high confidence” list were considered.

FIG. 63 is a venn diagram illustrating the overlap of mRNA ψ sites between BACS and BID-seq.

FIG. 64 shows the distribution of BID-seq-only ψ sites in BACS dataset.

FIG. 65 shows a venn diagram illustrating the overlap of mRNA ψ sites between BACS and PRAISE.

FIG. 66 shows the distribution of PRAISE-only ψ sites in BACS dataset.

### DETAILED DESCRIPTION

The inventors in seeking to improve on existing methods of pseudouridine detection and sequencing have developed a 2-BromoAcrylamide-assisted Cyclization Sequencing (BACS) method for direct and base-resolution sequencing of ψ.

BACS provides quantitative and base-resolution sequencing of ψ. BACS uses bromoacrylamide cyclization chemistry and induces ψ-to-C mutation signatures rather than truncation or deletion signatures, allowing for more accurate quantification of W stoichiometry. Importantly, BACS offers higher resolution compared with CMC-based methods, particularly in highly structured regions. Moreover, BACS overcomes the inherent limitations of BS-based methods in two crucial aspects: it enhances the detection of densely modified ψ sites with higher accuracy and sensitivity, and it facilitates the precise determination of the exact position of ψ sites located adjacent to one or more uridines. These advancements make BACS a valuable tool for studying and understanding ψ modifications in cellular RNAs, as it can provide a more comprehensive and accurate picture of the ψ landscape across various RNA species. Using BACS, the inventors have successfully generated the first quantitative ψ map of human snoRNA and tRNA, shedding light on the distribution of ψ modifications in small RNAs. The combination of BACS with the latest techniques has the potential to further improve its performance on small RNAs. The reliability and robustness of BACS are reaffirmed when applied to mRNA, as it consistently produced results in line with reported datasets. BACS is therefore a powerful method for studying ψ modifications.

BACS will permit exploration of variation of ψ across different cell types. BACS, with its minimal RNA degradation, presents an excellent opportunity to be combined with single-cell techniques, which could enable the study of ψ dynamics in diverse cell populations. The BACS method of the invention which can realize quantitative and base-resolution sequencing of ψ may be used to investigate the pseudouridylation of nascent RNA. Also, there are the 13 putative PUS enzymes in the human genome and the identification of the PUS enzyme responsible for many ψ sites remains challenging. Moreover, their substrate specificity and potential redundancy are not fully understood. BACS can serve as a valuable tool for studying PUS knockout cells to elucidate the properties and functions of these enzymes.

As used herein, the term Cₘ-Cₙ refers to a group with m to n carbon atoms. For the absence of doubt, the term “Co” refers to a group with 0 carbon atoms.

The term “alkyl” refers to a monovalent linear or branched saturated hydrocarbon chain. For example, C₁-C₆-alkyl may refer to methyl, ethyl, n-propyl, iso-propyl, n-butyl, sec-butyl, tert-butyl, n-pentyl and n-hexyl. The alkyl groups may be unsubstituted or substituted by one or more substituents.

The term “alkylene” refers to a bivalent linear saturated hydrocarbon chain. For example, C₁-C₃-alkylene may refer to methylene, ethylene or propylene. The alkylene groups may be unsubstituted or substituted by one or more substituents. For the absence of doubt, the term “C₀-alkylene” refers to a group in which an alkylene chain is absent. For example, “C₀-alkylene-R^{a}” refers to an R^{a}.

The term “haloalkyl” refers to a hydrocarbon chain substituted with at least one halogen atom independently chosen at each occurrence from: fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be a fluoroalkyl group, i.e. a hydrocarbon chain substituted with at least one fluorine atom. Thus, a haloalkyl group may have any amount of halogen substituents. The group may contain a single halogen substituent, it may have two or three halogen substituents, or it may be saturated with halogen substituents.

The term “alkenyl” refers to a branched or linear hydrocarbon chain containing at least one double bond. The double bond(s) may be present as the E or Z isomer. The double bond may be at any possible position of the hydrocarbon chain. For example, “C₂-C₆-alkenyl” may refer to ethenyl, propenyl, butenyl, butadienyl, pentenyl, pentadienyl, hexenyl and hexadienyl. The alkenyl groups may be unsubstituted or substituted by one or more substituents.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. The alkynyl groups may be unsubstituted or substituted by one or more substituents.

The term “cycloalkyl” refers to a saturated hydrocarbon ring system containing 3, 4, 5 or 6 carbon atoms. For example, “C₃-C₆-cycloalkyl” may refer to cyclopropyl, cyclobutyl, cyclopentyl, cyclohexyl. The cycloalkyl groups may be unsubstituted or substituted by one or more substituents.

The term “y- to z-membered heterocycloalkyl” refers to a y- to z-membered heterocycloalkyl group. Thus it may refer to a monocyclic or bicyclic saturated or partially saturated group having from y to z atoms in the ring system and comprising 1 or 2 heteroatoms independently selected from O, S and N in the ring system (in other words 1 or 2 of the atoms forming the ring system are selected from O, S and N). By partially saturated it is meant that the ring may comprise one or two double bonds. This applies particularly to monocyclic rings with from 5 to 6 members. The double bond will typically be between two carbon atoms but may be between a carbon atom and a nitrogen atom. Examples of heterocycloalkyl groups include; piperidine, piperazine, morpholine, thiomorpholine, pyrrolidine, tetrahydrofuran, tetrahydrothiophene, dihydrofuran, tetrahydropyran, dihydropyran, dioxane, azepine. A heterocycloalkyl group may be unsubstituted or substituted by one or more substituents.

Aryl groups may be any aromatic carbocyclic ring system (i.e. a ring system containing 2(2n+1)π electrons). Aryl groups may have from 6 to 10 carbon atoms in the ring system. Aryl groups will typically be phenyl groups. Aryl groups may be naphthyl groups or biphenyl groups.

The term ‘heterocyclyl’ group refers to rings comprising from 1 to 4 heteroatoms independently selected from O, S and N. The rings may be heterocycloalkyl rings (including both saturated and partially saturated rings) or heteroaryl rings. The term “heterocyclyl” also encompasses groups that are tautomers of hydroxy heteroaryl groups, such pyridones, and tautomers of hydroxy heteroaryl groups that are substituted on the nitrogen, such as N-alkyl pyridones.

The term ‘heterocycloalkenyl’ refers to partially saturated rings comprising from 1 to 2 heteroatoms independently selected from O, S and N.

The term “heteroaryl” refers to any aromatic (i.e. a ring system containing 2(2n+1)π electrons) ring system comprising from 1 to 4 heteroatoms independently selected from O, S and N (in other words from 1 to 4 of the atoms forming the ring system are selected from O, S and N). Thus, any heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g. 1-2) nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine, quinoline, isoquinoline, indole, benzofuran, benzopyrazole, benzimidazole.

### EXAMPLES

### Methods

Preparation of model RNA. Regular and W-labeled 10mer RNA oligonucleotides and 30mer spike-ins were purchased from Integrated DNA Technologies (IDT). The 72mer W-containing model RNA used for mutation analysis and the 1.8-kb 10% ψ-modified RNA used for UHPLC-MS/MS were prepared by T7 in vitro transcription using HiScribe T7 High Yield RNA Synthesis Kit (New England Biolabs (NEB)) and Pseudo-UTP (Jena Bioscience) according to the manufacturer's protocol. The DNA template was removed by adding 2 μl Turbo DNase (Invitrogen) to the reaction and incubating at 37° C. for 30 min. The products were finally purified with Monarch RNA Cleanup Kit (NEB). Sequences of RNA oligonucleotides can be found in Table 1 below:

TABLE 1

| Name | Sequence (5′ to 3′) | Source |
|---|---|---|
| for MALDI | | |
| 10 mer U-ORN | UACUGUAGCU | IDT |
| 10 mer Ψ-ORN | UACUGΨAGCU | IDT |
| for mutation analysis | | |
| 72 mer Ψ-ORN | GGGAGAACACACCACAACGAAAC | in vitro transcription |
| | CAACGGΨACAACAACAGAAAΨCG | Pseudo-UTP, ATP, |
| | AGGACCGAAGCGAAGGCAAAGAC | CTP, GTP |
| | AAC | |
| for UHPLC-MS/MS | | |
| 1.8-kb 10% Ψ- | T7 in vitro transcription using | in vitro transcription |
| modified RNA | linearized Fluc plasmid (NEB) | 10% Pseudo-UTP, |
| | as template | 90% UTP, ATP, |
| | | CTP, GTP |
| spike-ins | | |
| 30 mer NNUNN | AUGUCUCGACGUNNUNNGUUAC | IDT |
| | AGUACCGU | |
| 30 mer NNΨNN | GCUUCAAGUUGANNΨNNCAUCG | IDT |
| | CAAGUGCA | |

Mass spectrometry analysis of short oligonucleotides. MALDI was performed on a Voyager-DE Biospectrometry Workstation (Applied Biosystems) with 2′,4′,6′-trihydroxyacetophenone (THAP) as matrix. All the oligonucleotides were analyzed in positive mode.

Quantification of ψ level by UHPLC-MS/MS. The untreated and treated RNA were digested into nucleosides by Nucleoside Digestion Mix (NEB) in a 50 μl solution according to the manufacturer's protocol. After filtering with Amicon Ultra-0.5 mL 3K centrifugal filters (Millipore), the digested samples were subjected to UHPLC-MS/MS analysis as described in Muller, C. A. et al., Nat. Methods 16, 429-436 (2019). 1290 Infinity LC Systems (Agilent) was equipped with a ZORBAX RRHD SB-C18 column (2.1×150 mm, 1.8 μm, Agilent) coupled with a 6495B Triple Quadrupole Mass Spectrometer (Agilent). The ions were monitored in positive mode with mass transitions of m/z 245 to 125 (ψ+H) and m/z 245 to 113 (rU+H) according to the compound-dependent UHPLC-MS/MS parameters for nucleoside quantification as shown in table 2 below.

TABLE 2

| | Precursor | Product | RT | Delta RT | |
|---|---|---|---|---|---|
| Compound | Ion (m/z) | Ion (m/z) | (min) | (min) | CE (V) |
| Ψ + H | 245 | 125 | 1.8 | 2.0 | 10.0 |
| rC + H | 244 | 112 | 2.3 | 2.0 | 10.0 |
| rC + Na | 266 | 134 | 2.3 | 2.0 | 10.0 |
| rU + H | 245 | 113 | 3.3 | 2.0 | 10.0 |
| rG + H | 284 | 152 | 8.1 | 2.0 | 10.0 |
| rA + H | 268 | 136 | 12.9 | 2.0 | 8.0 |

RT: retention time; CE: collision energy.

Concentrations of nucleosides in RNA samples were deduced by fitting the signal peak areas into the standard curves.

Cell culture. HeLa cells were cultured in DMEM medium (Gibco) supplemented with 10% (v/v) FBS (Gibco) and 1% penicillin/streptomycin (Gibco) at 37° C. with 5% CO₂. For isolation of RNA, cells were harvested by centrifugation for 5 min at 1,000×g and room temperature.

RNA isolation. Total RNA was isolated using TRIzol (Invitrogen) and Direct-zol RNA Miniprep Plus (Zymo Research) according to the manufacturer's protocol. Ribo⁻ RNA was isolated using RiboMinus Eukaryote System v2 (Invitrogen) according to the manufacturer's protocol. PolyA⁺ RNA was isolated by two rounds of polyA-tailed selection using Dynabeads mRNA DIRECT Purification Kit (Invitrogen) according to the manufacturer's protocol. To remove genomic DNA contamination, RNA was then treated with Turbo DNase and purified by Zymo-IC Column with RNA binding buffer.

BACS for ψ detection. 50-100 ng ribo- or polyA+RNA was fragmented by NEBNext Magnesium RNA Fragmentation Module at 94° C. for 4 min according to the manufacturer's protocol and purified by Zymo-IC Column with RNA binding buffer. The fragmented RNA was mixed with 5 μl 10×T4 PNK reaction buffer (NEB), 5 μl T4 PNK (NEB), and 2.5 μl SUPERase. In RNase Inhibitor (Invitrogen) in a 50 μL final solution and incubated at 37° C. for 1 h. The 3′-repaired RNA was purified by Zymo-IC Column with RNA binding buffer and eluted with 10 μl nuclease-free H₂O. The eluted RNA was then mixed with 1 μl synthetic 30mer spike-ins (2%) and 1 μl of 20 μM RNA adapter (5′-/5rApp/AGATCGGAAGAGCGTCGTG/3SpC3/-3′), incubated at 70° C. for 2 min and immediately placed on ice. Next, 2.5 μl 10×T4 RNA Ligase reaction buffer (NEB), 1 μl SUPERase. In RNase Inhibitor, 7.5 μl 50% PEG 8000 (NEB), and 2 μl T4 RNA Ligase 2, truncated KQ (NEB) were added to the mixture and the reaction was incubated at 25° C. for 2 h followed by 16° C. for 14 h. To digest excess adapters, the solution was further diluted to 47 μl with nuclease-free H₂O and treated with 2 μl 5′-Deadenylase (NEB) at 30° C. for 1 h followed by adding 1 μl RecJr (NEB) and incubating at 37° C. for 1 h. The 3′-ligated RNA was purified by Zymo-IC Column with RNA binding buffer and eluted with 10 μl nuclease-free H₂O. A 7 μl aliquot was subjected to BACS library construction, while the rest 3 μl was saved as control sample and diluted to 12.5 μl with nuclease-free H₂O. For BACS, 1 M 2-bromoacrylamide (Enamine) was prepared by dissolving the solid in DMSO. 7 μl 3′-ligated RNA was added into a 20 μl solution containing 250 mM 2-bromoacrylamide and 625 mM phosphate buffer (pH 8.5) and incubated at 85° C. for 30 min. The treated RNA was double purified by Micro Bio-Spin P-6 Tris Column (Bio-Rad) and Zymo-IC Column with RNA binding buffer and finally eluted with 12.5 μl nuclease-free H₂O.

Both treated and control RNA samples were mixed with 1 μl of 2 μM RT primer (5′-ACACGACGCTCTTCCGATCT-3′) and 1 μl of 10 mM dNTP mix (NEB), incubated at 70° C. for 2 min and immediately placed on ice. Next, 4 μl 5×Maxima H⁻ RT buffer (Thermo), 0.5 μl RiboLock RNase Inhibitor (Thermo), and 1 μl Maxima H-Reverse Transcriptase (Thermo) were added to the mixture and the reaction was incubated at 50° C. for 1 h. To digest excess RT primers, the solution was treated with 1 μl Exo I (NEB) and incubated at 37° C. for 30 min followed by adding 1 μl of 0.5 M EDTA (Sigma) to quench the reaction. To hydrolyze the RNA, 2.5 μl of 1 M NaOH (Sigma) was added and the solution was then incubated at 70° C. for 12 min followed by adding 2.5 μl of 1 M HCl (Sigma) to neutralize NaOH. The cDNA was finally purified with Dynabeads MyOne Silane (Invitrogen) and eluted with 13 μl nuclease-free H₂O. The eluted cDNA was then mixed with 2 μl of 25 μM cDNA adapter (5′-/5Phos/NNNNNNAGATCGGAAGAGCACACGTCTG/3SpC3/-3′), incubated at 70° C. for 2 min and immediately placed on ice. Next, 5 μl 10×T4 RNA Ligase reaction buffer, 25 μl 50% PEG 8000, 0.5 μl of 100 mM ATP (NEB), 3.5 μl DMSO (Thermo), and 1 μl T4 RNA Ligase 1, high concentration (NEB) were added to the mixture and the reaction was incubated at 25° C. for 16 h. The ligated cDNA was purified with Dynabeads MyOne Silane and eluted with 15 μl nuclease-free H₂O. The eluted DNA was amplified with NEBNext Multiplex Oligos for Illumina (96 Unique Dual Index Primer Pairs) and NEBNext Ultra II Q5 Master Mix for 10 cycles according to the manufacturer's protocol. The PCR products were purified with 0.8×AMPure XP beads and quantified with Qubit dsDNA HS Assay Kit (Thermo) according to the manufacturer's protocol. BACS and control libraries were sequenced on NextSeq 2000 (60 bp paired end) with no PhiX added.

Data pre-processing. Raw sequencing reads were processed by Cutadapt v.4.2 (Martin, M. EMBnet.journal 17, 10-12 (2011)) to remove low-quality bases (−q 20) and short reads (−m 18), as well as to trim adaptors. 6mer UMI were extracted by UMI-tools extract v.1.0.1 (Smith, T., et al., Genome Res. 27, 491-499 (2017)) and used for deduplication. Paired reads were then merged into single reads using fastp v.1.0.1 (Chen, S. F., et al., Bioinformatics 34, 884-890 (2018)).

Read alignment. Cleaned reads were first mapped to synthetic spike-ins and rRNA references using bowtie2 v.2.4.4 (Langmead, B. & Salzberg, S. L. Nat. Methods 9, 357-359 (2012)). The key parameters are as follows: bowtie2 -p 2 -no-unal -local -L 16 -N 1 -mp 4. The unaligned reads were subsequently mapped to snoRNA references and then to tRNA references, using the same parameters. Human snoRNA sequences that belong to HGNC “Small nucleolar RNAs” gene group were downloaded from RefSeq. Duplicate snoRNA sequences were removed. High-confidence human tRNA sequences (hg38) were downloaded from GtRNAdb (Chan, P. P. & Lowe, T. M. Nucleic Acids Res. 44, D184-D189 (2016)). Only non-redundant tRNA sequences were kept and appended with a “3′-CCA” end. Finally, unmapped reads were aligned to human genome (hg38) with GENCODE v.43 annotation by STAR v.2.7.9a (Dobin, A. et al., Bioinformatics 29, 15-21 (2013)). The aligned reads were then filtered and sorted using samtools v.1.16.1 (Li, H. et al., Bioinformatics 25, 2078-2079 (2009)). For synthetic spike-ins and rRNA, only reads with MAPQ ≥10 were kept. For snoRNA and tRNA, only reads with MAPQ ≥1 were kept. For mRNA, only uniquely mapped reads (−q 30) with a maximum of 3 mutation counts were kept. Deduplication was performed using UMI-tools dedup v.1.0.1 Smith, T., et al., Genome Res. 27, 491-499 (2017)). Additionally, poly-C counts (more than 3 cytidines) at the beginning and end of STAR-aligned reads were trimmed using GATK ClipReads (v.4.1.7.0)⁸¹ to avoid potential false-positive signals. Finally, mutations are counted by samtools mpileup v.1.16.1 (McKenna, A. et al. Genome Res. 20, 1297-1303 (2010)) and cpup (v.0.1.0) (https://github.com/y9c/cpup).

Calling 4V site. BACS raw conversion rates were calculated as C/(T+C). The W modification levels were calculated using the linear equation: ψ modification level=(R−F)/(C−F), where R, F, and C indicated raw conversion rates, motif-specific false-positive rates (from NNUNN spike-in), and motif-specific conversion rates (from NNψNN spike-in), respectively. A p-value was calculated for each site using the motif-specific false-positive rates and then adjusted following the Benjamini-Hochberg (BH) procedure. The following criteria were used to call ψ sites: (1) coverage higher than 20 in both BACS and control libraries; (2) background conversion rates lower than 0.01 or T-to-C mutation counts less than 2 in control libraries; (3) ψ modification level higher than 0.05; (4) adjusted p-value lower than 0.001; (5) consistently detected in all replicates. For calling cy-tRNA ψ sites, criteria (3) and (5) were modified to require a ψ modification level higher than 0.10 in at least two out of three replicates. Only ψ sites identified in expressed cy-tRNA isodecoders were reported.

RNA structure visualization. The RNA-RNA interactions were visualized using r2r v.1.0.6 (Weinberg, Z. & Breaker, R. R. BMC Bioinformatics 12, 3 (2011)). The snoRNA-rRNA interactions were adapted from snoRNA Atlas.

Downstream analysis. The snoRNA box and guide sequences were downloaded from snoDB 2.0 (Bergeron, D. et al., Nucleic Acids Res. 51 (2022)). In the metagene analysis, snoRNA sequences that displayed considerable similarity were streamlined, retaining only one representative snoRNA. The annotation of ψ sites identified in polyA-tailed RNA was performed using bedtools intersect v.2.30.0 (Quinlan, A. R. & Hall, I. M. Bioinformatics 26, 841-842 (2010)) with GENCODE v.43 annotation. ψ sites in regions of interest were visualized by Integrative Genomics Viewer (IGV) (Robinson, J. T. et al., Nat. Biotechnol. 29, 24-26 (2011).

Read counts obtained from featureCounts v.1.6.4 (Liao, Y., et al., Bioinformatics 30, 923-930 (2014)) were normalized based on sequencing depth and gene length using the transcripts per million (TPM) method. GO analysis was performed with mRNA ψ sites using enrichR (Kuleshov, M. V. et al. Nucleic Acids Res. 44, W90-W97 (2016)).

Published data. Related published data were downloaded from the Gene Expression Omnibus (GEO) database: BID-seq for HeLa cells (GSE179798) (Dai, Q. et al., Nat. Biotechnol. 41, 344-354 (2023)).

### Example 1: Reaction of Oligonucleotide with 2-Bromoacrylamide

10mer short ψ- and U-labelled RNA oligonucleotides for MALDI were purchased from IDT. The oligonucleotide comprising a pseudouridine, 5′-UACUGψAGCU-3′ [SEQ ID NO: 1], was reacted with 2-bromoacrylamide. A control oligonucleotide lacking pseudouridine, 5′-UACUGUAGCU-3′ [SEQ ID NO: 2], was reacted separately with 2-bromoacrylamide at the same time and under the same conditions.

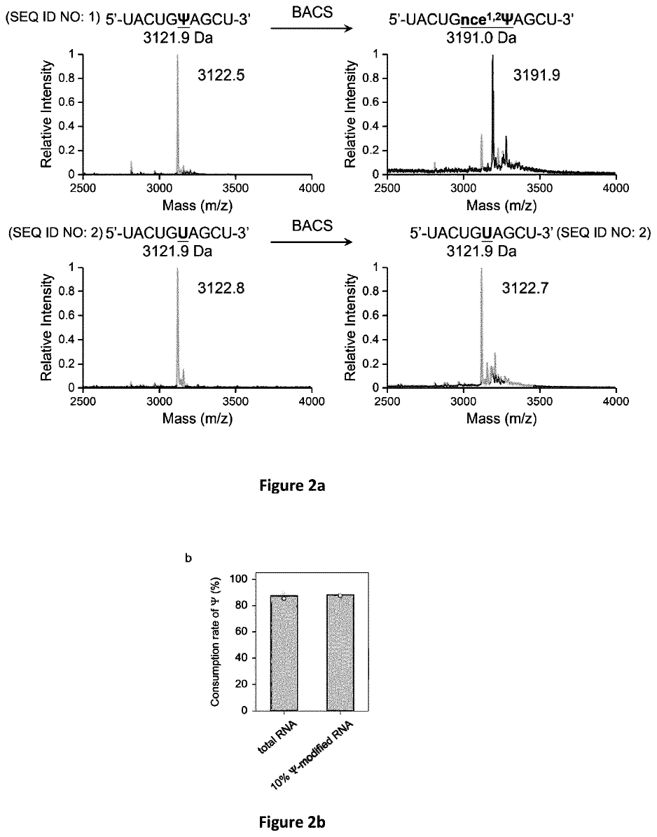

The reaction products of the experiment and control were then analysed by MALDI. As shown in FIG. 2a, the 3122.5 Da observed mass of the ψ oligonucleotide increased after the reaction to 3191.9 Da observed mass and so an increase in mass of 69 Da was found. In comparison the control oligonucleotide showed no apparent increase in mass. The calculated mass of each reaction product is shown immediately below its respective sequence. The increase of mass values was found for the pseudouridine containing oligonucleotide supports the formation of a cyclization product (carbamido-1, O²-ethano ψ, nce^{1,2}ψ). This reaction was further confirmed by ultra-high-performance liquid chromatography-tandem mass spectrometry (UHPLC-MS/MS) (see FIG. 2b).

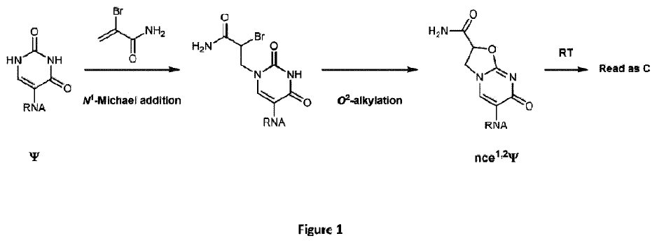

Comparing ψ with U, ψ contains one free N1 atom, which is found to be highly reactive towards Michael addition acceptors (such as acrylonitrile, acrylamide, and other acrylic compounds). With reference to FIG. 1, the presence of an α-halogen group on the acceptors is found to cause tandem cyclization of N1-acrylic adduct of ψ through O²-intramolecular alkylation, thus inducing the desired ψ-to-C mutation.

### Example 2: Determination of the U-to-C Mutation Profile of Nce^{1,2}ψ

72mer in vitro transcribed ψ/U-containing RNA was used to validate the ψ-to-C mutation profile of nce^{1,2}ψ. For preparation of model RNA and spike-ins, 72mer ψ- and U-containing RNA oligonucleotides were synthesized by T7 in vitro transcription using HiScribe® T7 High Yield RNA Synthesis Kit (NEB) and Pseudo-UTP (Jena) or UTP, along with ATP, CTP, and GTP according to the manufacturer's protocol. The template DNA were removed by adding 2 μl Turbo™ DNase (Thermo) into the reaction and incubating at 37° C. for 30 min. The template DNA sequence used was:

[SEQ ID NO: 3]
5′-GTTGTCTTTGCCTTCGCTTCGGTCCTCGATTTCTGTTGTTGTACCG
TTGGTTTCGTTGTGGTGTGTTCTCCCTATAGTGAGTCGTATTA-3′

The final RNA sequence was:

[SEQ ID NO: 4]
5′-GGGAGAACACACCACAACGAAACCAACGG(Ψ/U)ACAACAACAGA
AA(Ψ/U)CGAGGACCGAAGCGAAGGCAAAGACAAC-3′

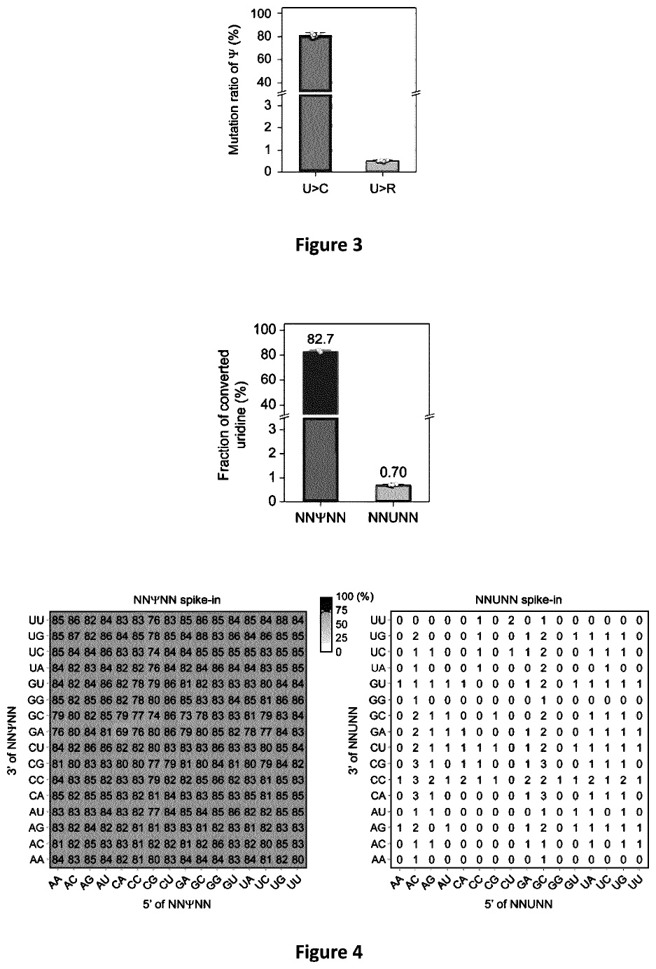

The products were finally purified with Monarch® RNA Cleanup Kit (NEB). Through sequencing, 80% U-to-C mutation rates were observed on the two sites, while U-to-R (R=A or G) mutation rates were lower than 1% (see FIG. 3). Therefore, the U-to-C mutation rate can serve as the conversion rate of BACS.

### Example 3: Sequence Preference of BACS

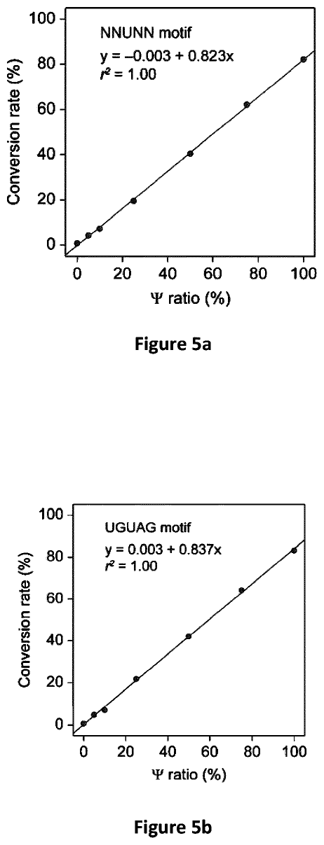

To further demonstrate the sequence preference of BACS, libraries were generated with synthetic 30mer RNA spike-in containing NNψNN and NNUNN (N=A, C, G or U), respectively. As shown in FIG. 4, after BACS there was an 82.7% conversion rate of ψ and a 0.7% false-positive rate of uridine when accumulating all motifs. Among all the 256 motifs, 224 of them showed a conversion rate higher than 80% and 255 of them displayed a conversion rate higher than 70%, suggesting the high efficiency of BACS chemistry. A low false-positive rate (<1%) was observed in most motifs (214 out of 256 motifs). Certain motifs, especially those with one or more cytidines 5′- or 3′-flanking to the uridine site (for example, GCUCC and ACUCC), displayed slightly higher false-positive rates (3%), possibly due to the preferences of RT. Nevertheless, BACS clearly showed higher conversion rates and lower false-positive rates than BID-seq both in general and in specific motifs. FIGS. 5a and 5b show that by mixing NNψNN and NNUNN spike-in in different ratios, excellent calibration curves were generated for accurate quantification of W modification level (r²=1.00).

### Example 4: Validation of BACS on Human rRNA

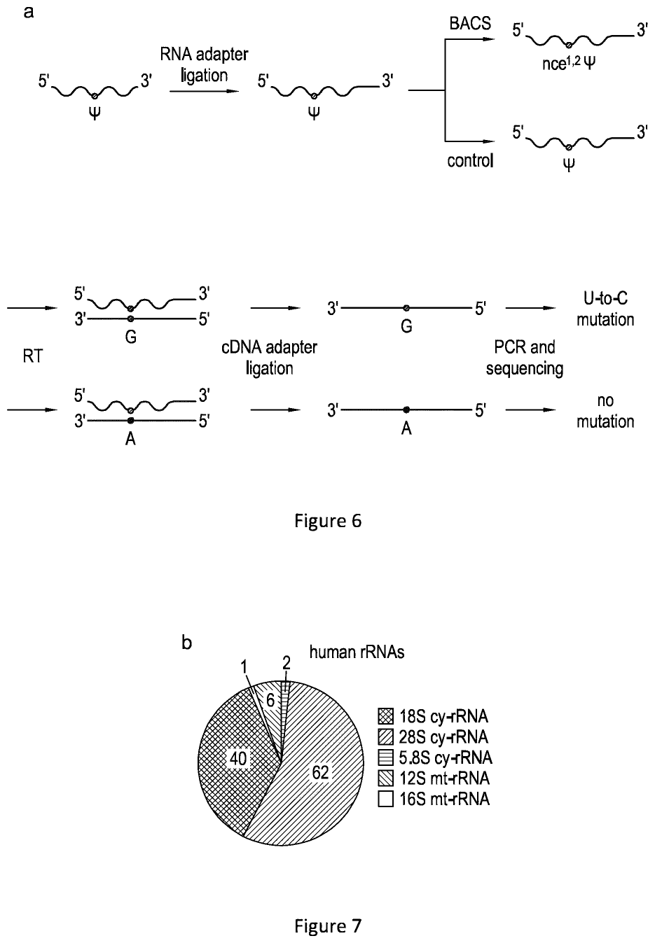

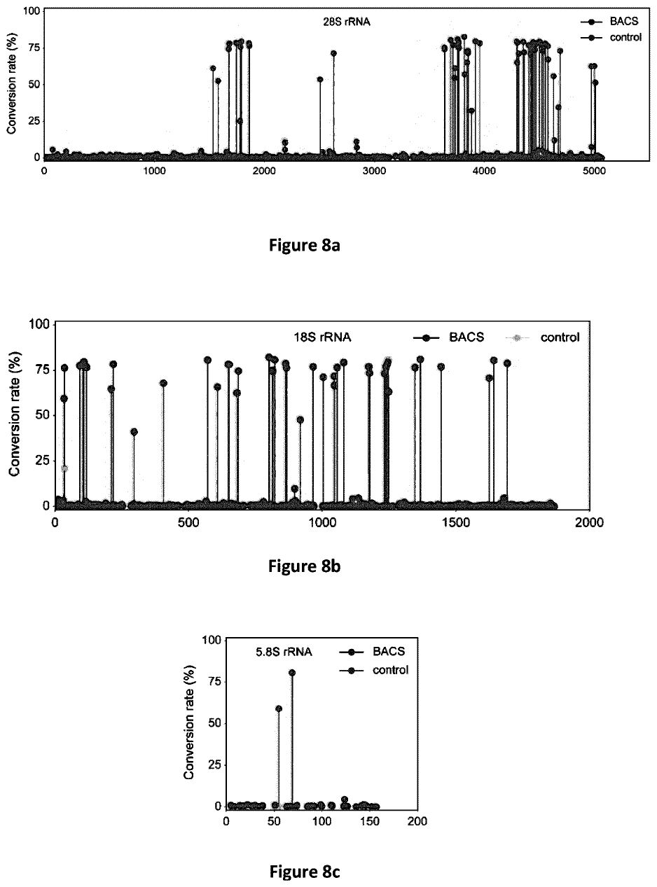

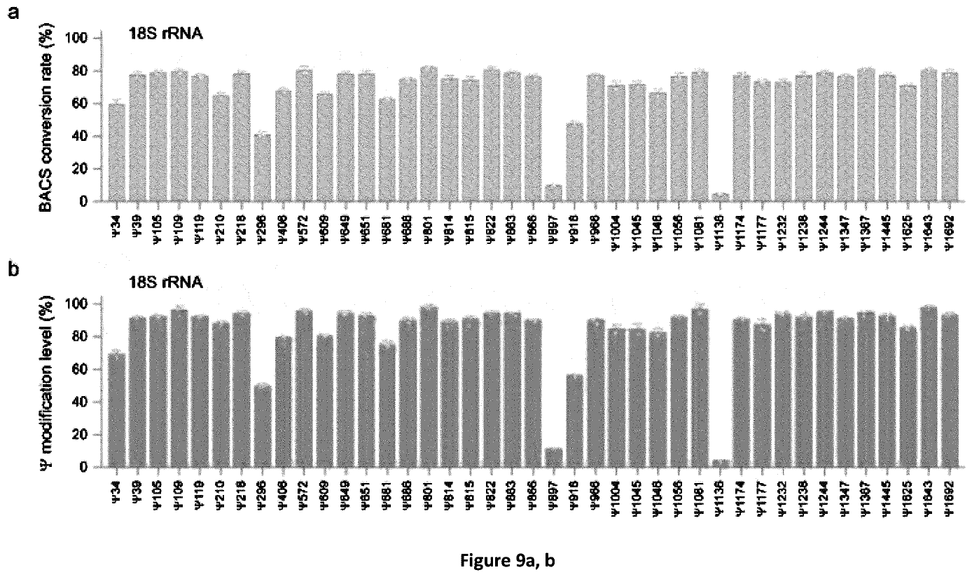

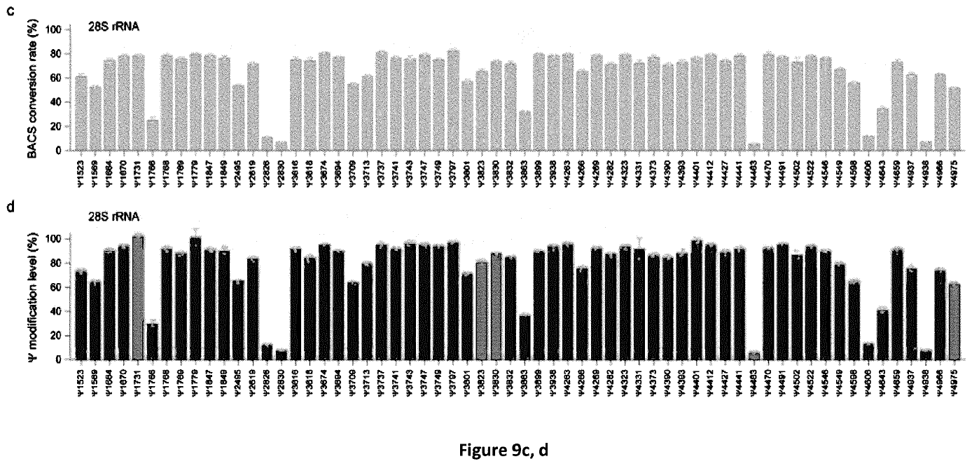

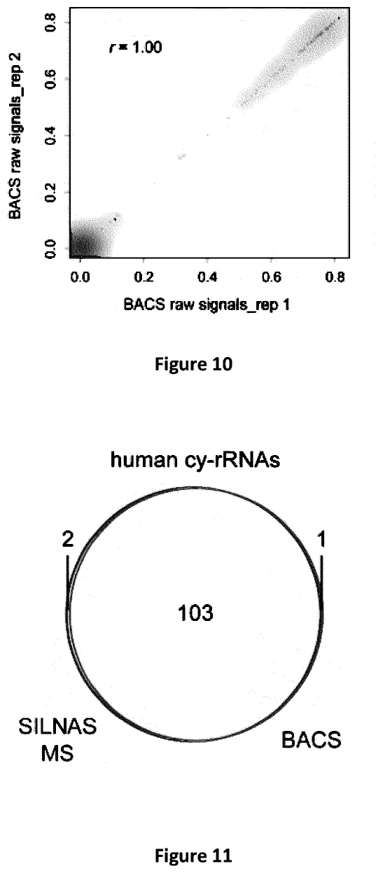

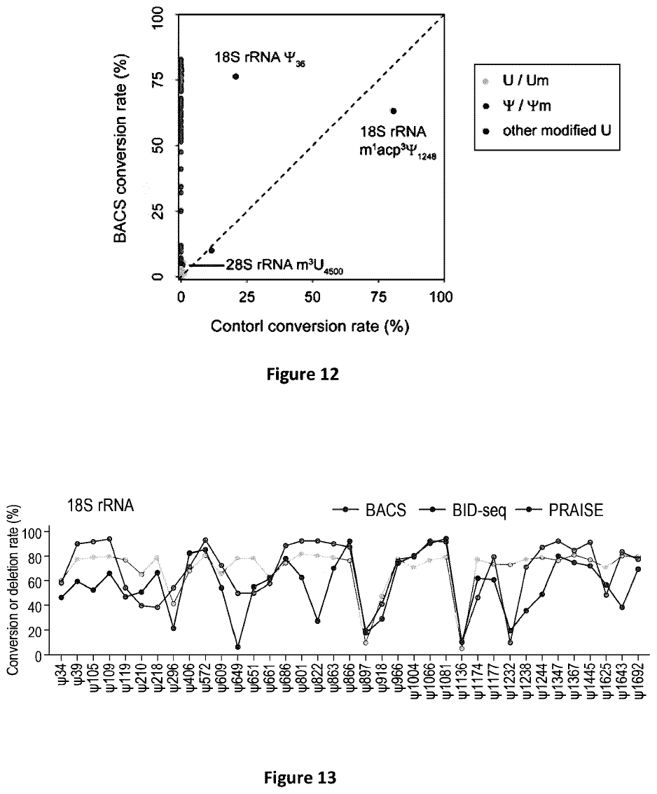

BACS was applied to cytosolic rRNA (cy-rRNA) from HeLa cells, which is known to possess a series of highly conserved ψ sites. FIG. 6 shows the workflow of library generation. FIG. 7 shows Based on the U-to-C mutation signals induced by BACS, 2, 40, and 62 ψ sites were detected in 5.8S, 18S, and 28S rRNAs, respectively. Most of the detected sites displayed a high modification level (>80%), consistent with the fact that W sites are highly modified in human cy-rRNA43 (see FIGS. 8a, 8b, 8c, 9a, 9b, 9c and 9d). Raw signals of BACS from two biological replicates were examined. As shown in FIG. 10, this revealed a strong correlation between them (Pearson's r=1.00 for two biological replicates. Compared with the reported SILNAS mass spectrometry (SILNAS MS) results, 103 out of 105 known ψ sites in human cy-rRNA (including one ψm site in 28S rRNA) were identified with high confidence (see FIG. 11). However, ψ1136 in 18S rRNA was not detected, possibly due to its low modification level of 3.8% by BACS (see FIGS. 9a and 9b). Interestingly, a 20% U-to-C mutation rate was found for the known 18S rRNA ψ36 site in control libraries, although the mutation rate increased to 75% after BACS treatment (see FIG. 8b and FIG. 12). Similar results were obtained from BID-seq control libraries, implying that there might be an uncharacterized single nucleotide polymorphism (SNP) site. In addition, a new ψ4938 site was detected in 28S rRNA, located adjacent to the previously known ψ4937 site. The presence of ψ4938 was supported by two public databases, both of which predicted that small nucleolar RNA (snoRNA) SNORA17B would be responsible for catalyzing this modification (see Jorjani, H. et al. Nucleic Acids Res. 44, 5068-5082 (2016) and Tan, K. T., et al., Sci. Adv. 7, eabd2605 (2021)). BACS therefore provided further confirmation of the existence of the ψ4938 site. It is important to note that while some ψ or uridine modifications can induce intrinsic mutation signals (such as U-to-C mutation for m1acp3ψ1248 in 18S rRNA and U-to-A mutation for m3U4500 in 28S rRNA), these can be easily filtered out by comparing the results of BACS libraries with control libraries (see FIG. 12).

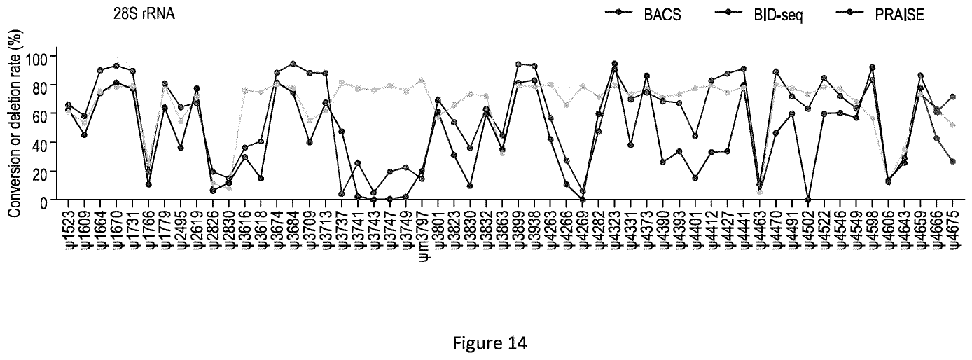

As expected, BACS clearly outperformed BS-based methods in the following aspects. First, the U-to-C mutation signature enabled BACS to determine the exact position of ψ sites in consecutive uridine sequences (adjacent to one or more uridines (for instance, ψ801/ψ814/ψ815/ψ822 in 18S rRNA and ψ1847/ψ1849 in 28S rRNA) and dense ψ sites in a narrow region (for example, ψ3737/ψ3741/ψ3743/ψ3747/ψ3749 in 28S rRNA and ψ4263/ψ4266/ψ4269 in 28S rRNA), while both remain challenging for BS-based methods (FIGS. 8a, 8b, 13 and 14). More even conversion rates of ψ sites were obtained across different regions of rRNA using BACS compared with BS-based methods, suggesting that BACS results would not be significantly influenced by the density of pseudouridylation and therefore enabled more accurate quantification of ψ stoichiometry (see FIGS. 13 and 14). Furthermore, BACS was found to be able to achieve a higher conversion rate on 28S rRNA ψm3797 site (85%) than BS-based methods (10-20%), because BACS solely relied on the availability of N1 atom of ψ (see FIG. 14).

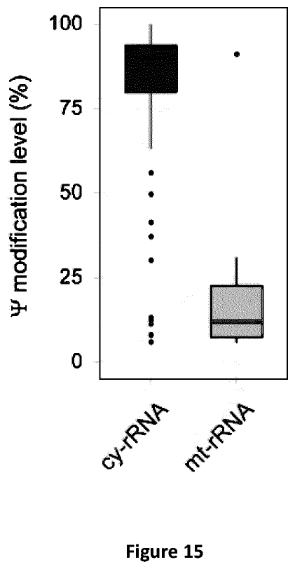

In addition to cy-rRNA, BACS was also applied to mitochondrial rRNA (mt-rRNA) and detected 6 and 1 ψ sites in 12S and 16S rRNAs, respectively. Among them, 4 sites have also been detected by Pseudo-seq4. In general, the modification level of ψ sites in mt-rRNA was significantly lower than their cytosolic counterparts (see FIG. 15).

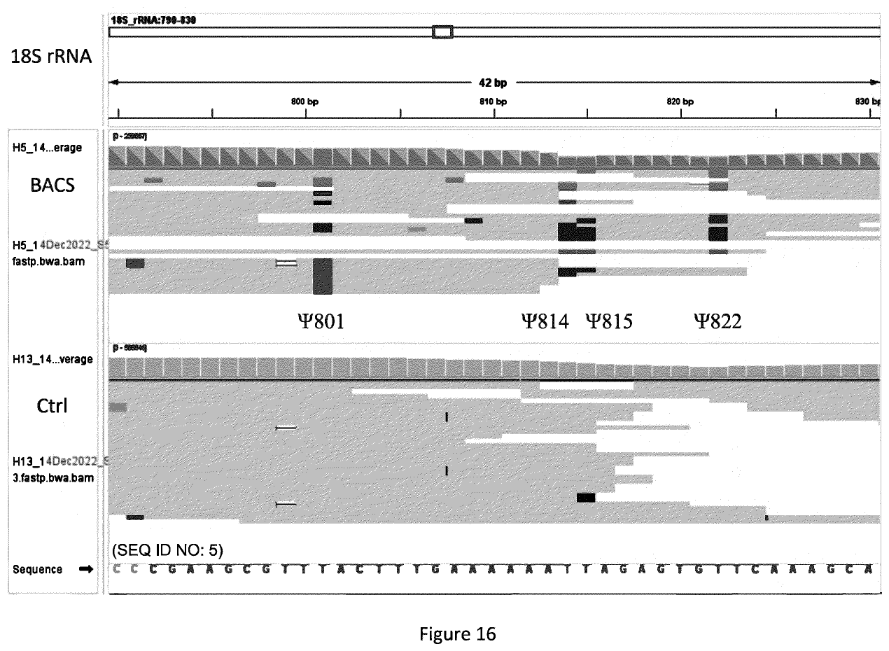

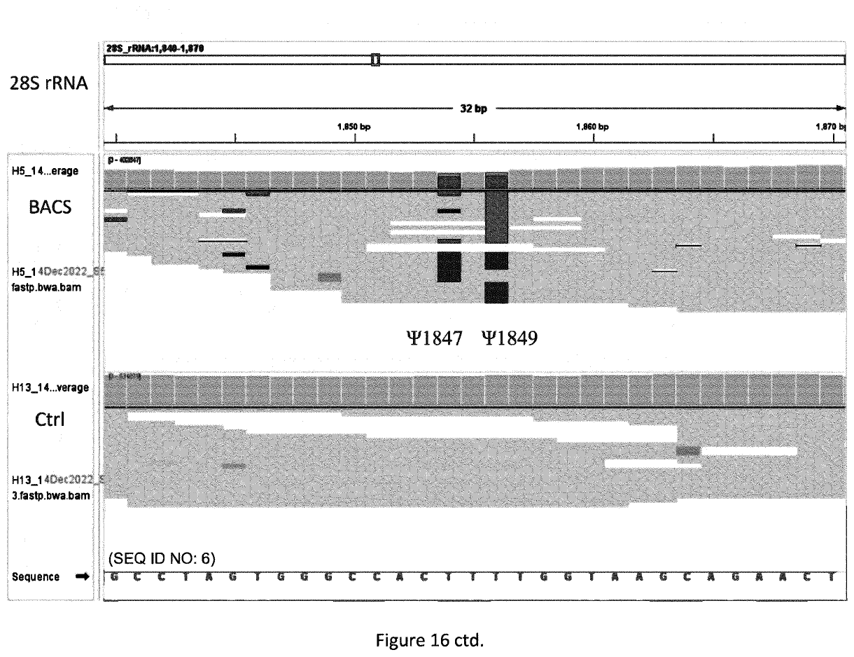

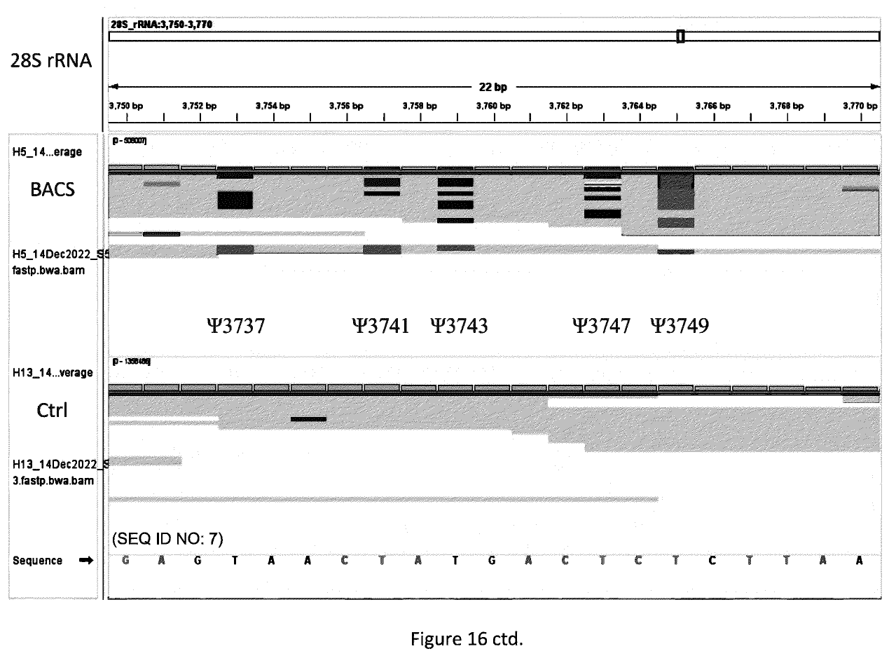

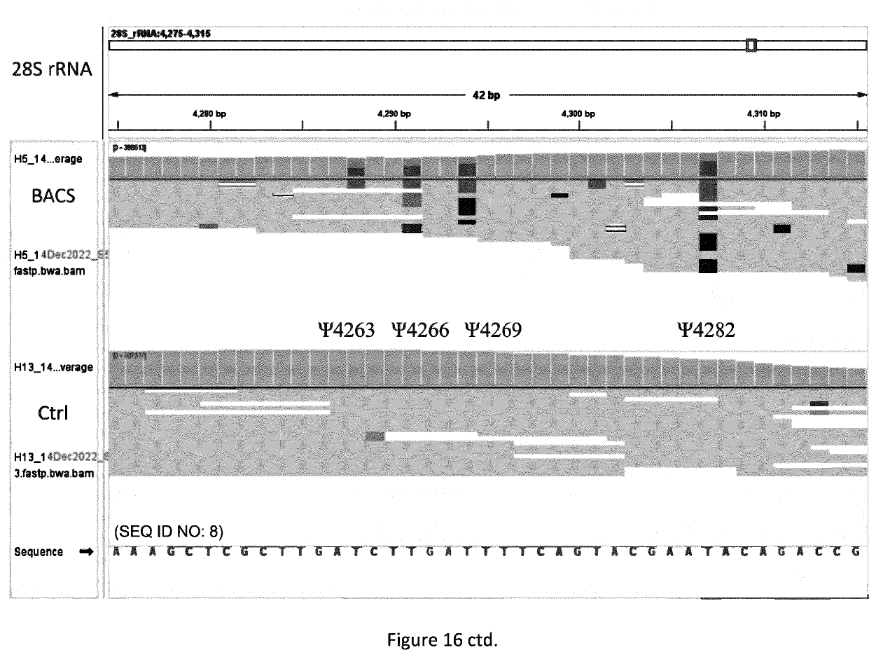

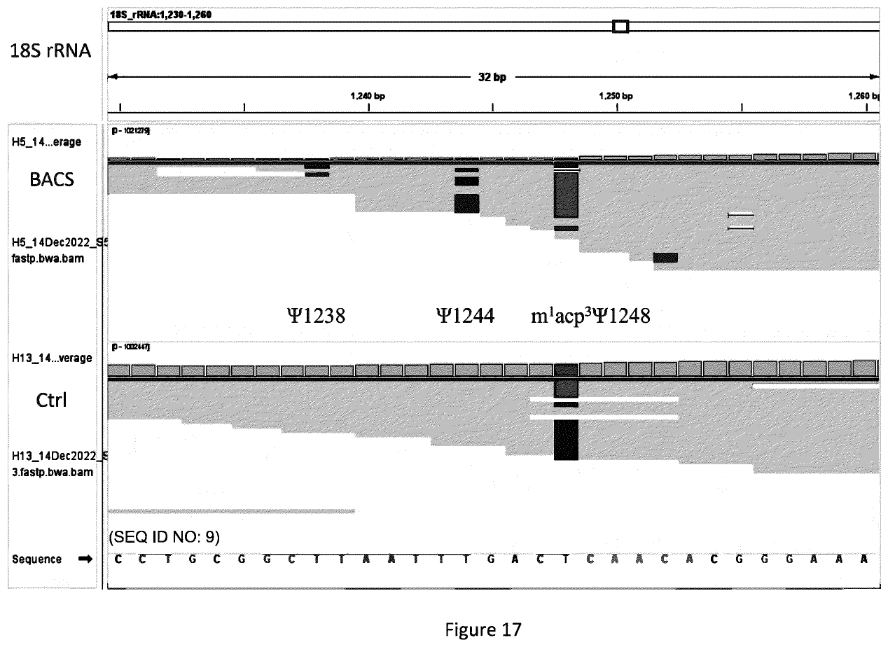

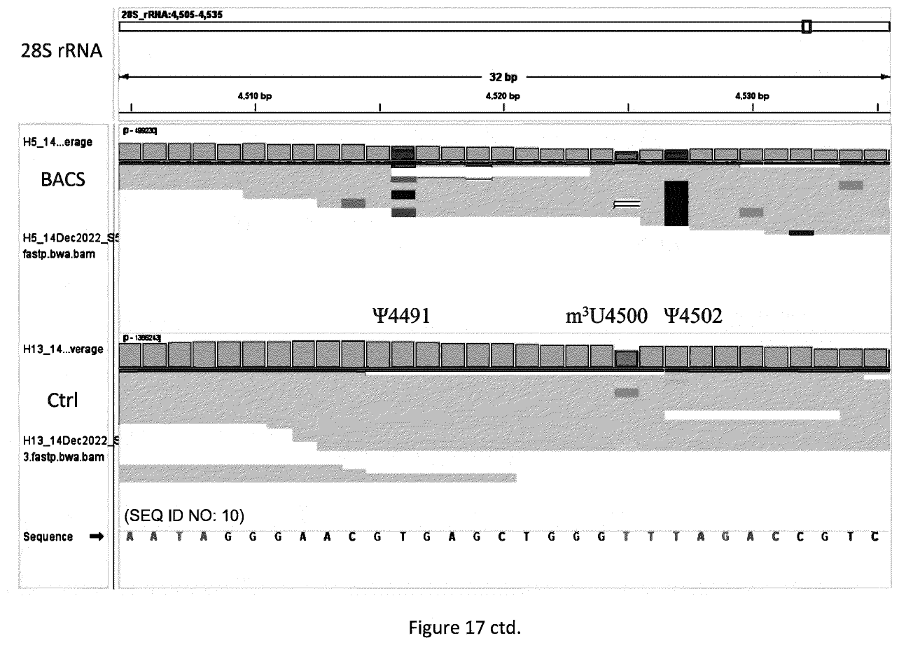

As shown in FIGS. 16 and 17, the mutation and deletion profiles induced by BACS treatment were checked for each base (A, C, G, U and ψ). Generally, BACS displayed high mutation rate on known ψ sites and low backgrounds on unmodified A, C, G and U sites. Moreover, the U-to-C mutation was confirmed to be the major type of Y mutation and thus could be used to calculate the conversion rate of Y. BACS does not result in significant deletion signature on ψ or other bases and this solves a fundamental problem in BS-based methods. As can be seen from FIG. 16, the mutation signature has enabled exact detection of ψ sites in the vicinity of one or more U (for instance, 18S rRNA ψ801, ψ822 and 28S rRNA ψ1847, ψ1849), or dense ψ sites in a narrow region (18S rRNA ψ801, ψ814, ψ815, ψ822; 28S rRNA ψ3737, ψ3741, ψ3743, ψ3747, ψ3749; ψ4263, ψ4266, ψ4269, ψ4282). Although some ψ or U modification would induce intrinsic mutation signature (U-to-C mutation for 18S rRNA m1acp3ψ1248 and U-to-A mutation for 28S rRNA m3U4500), FIG. 17 shows that these sites are readily excluded by comparing the BACS results with untreated RNA-seq data.

### Example 5: BACS Identified Highly Conserved ψ Sites in Human Spliceosomal snRNAs

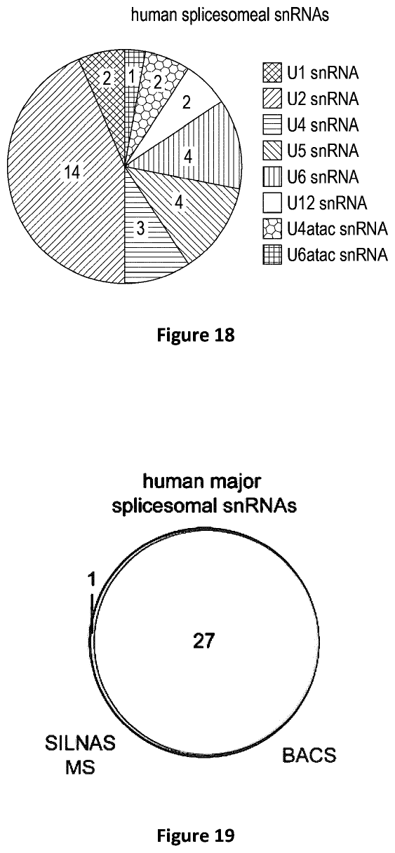

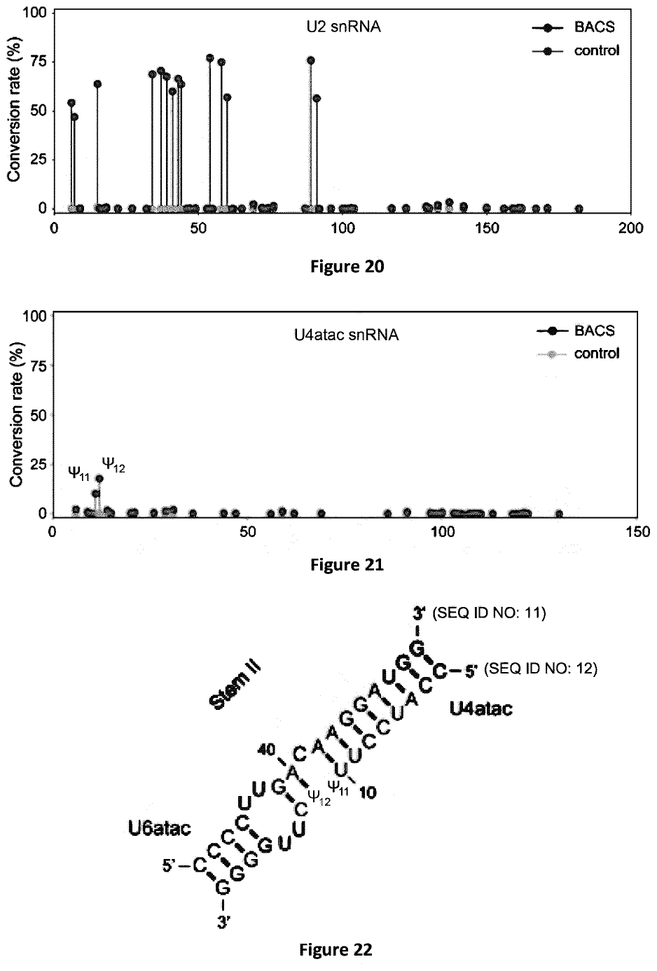

BACS was validated by applying it to spliceosomal snRNAs from HeLa cells, which is known to contain multiple consecutive ψ sites. The initial focus was on major spliceosomal snRNA species. 2, 14, 3, 4, and 4 ψ sites were detected in U1, U2, U4, U5, and U6 snRNAs, respectively, (see FIGS. 18 and 19) which is highly consistent with the latest SILNAS MS results of Yamaki, Y. et al., Anal. Chem. 92, 11349-11356 (2020). Only ψ₅₉ in U4 snRNA was not detected by BACS, since it is likely to be lowly modified in HeLa cells. It is noteworthy that BACS successfully mapped all 14 ψ sites in human U2 snRNA, which has not been realized by any other high-throughput sequencing methods, further demonstrating the superiority of BACS in detecting dense and consecutive ψ sites (see FIG. 20). Unlike the snRNA components of human major spliceosome, the ψ profile of minor spliceosomal snRNA species has only been revealed using CMC-based primer extension assay, mainly due to their low abundance. However, given that CMC-based methods may suffer from partial labeling efficiency and ‘stuttering’ phenomenon, we believed that BACS could be a better approach to study pseudouridylation in these snRNA species. Indeed, 2, 2, and 1 ψ sites were consistently detected in U12, U4atac, and U6atac snRNAs, respectively, while no ψ site was detected in U11 snRNA (see FIG. 18). Notably, two consecutive ψ sites (ψ₁₁/ψ₁₂) were confirmed rather than one ψ₁₂ site in U4atac snRNA, providing new insights into its interactions with U6atac snRNA (see FIGS. 21 and 22).

Additionally, conserved ψ₂₄₇ and ψ₂₅₀ sites were detected in in 7SK RNA and revealed that there was no high-confidence ψ site in U7 snRNA, RNase P RNA, RNase MRP RNA, vault RNA, and ψ RNA. However, the known ψ₂₁₁ site was not detected in 7SL RNA, possibly due to the differences of cell lines.

### Example 6: BACS Reveals the ψ Profile of Human snoRNA

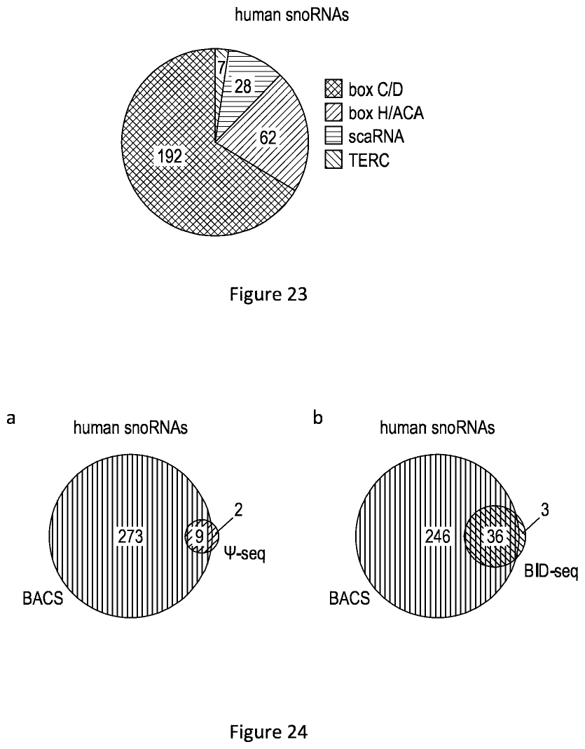

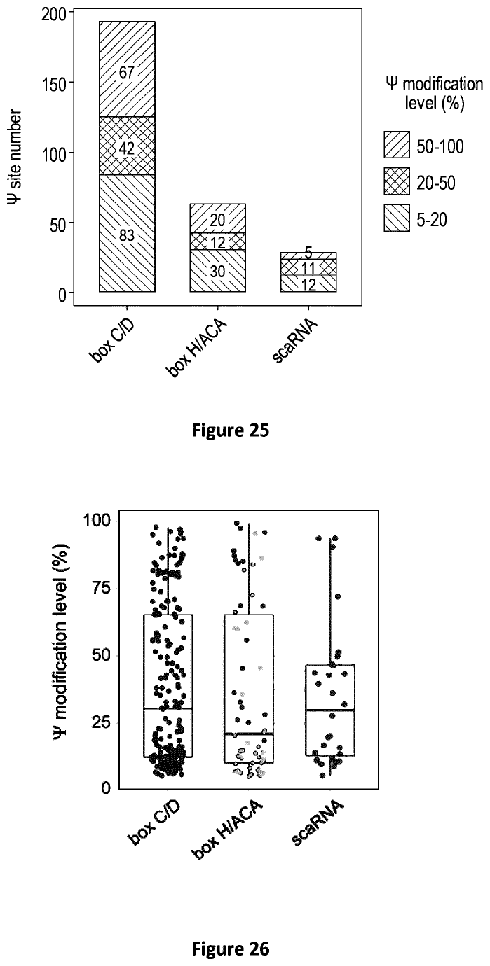

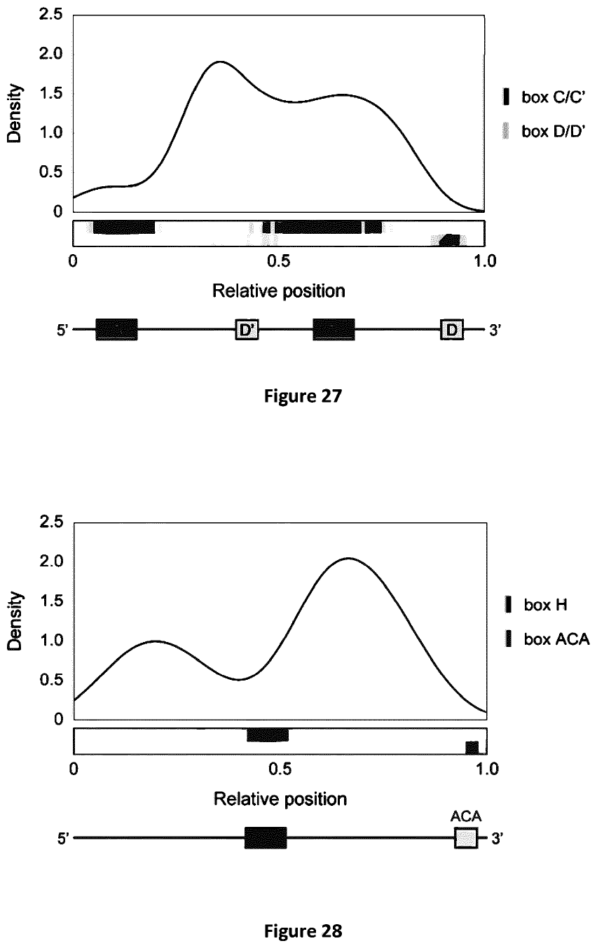

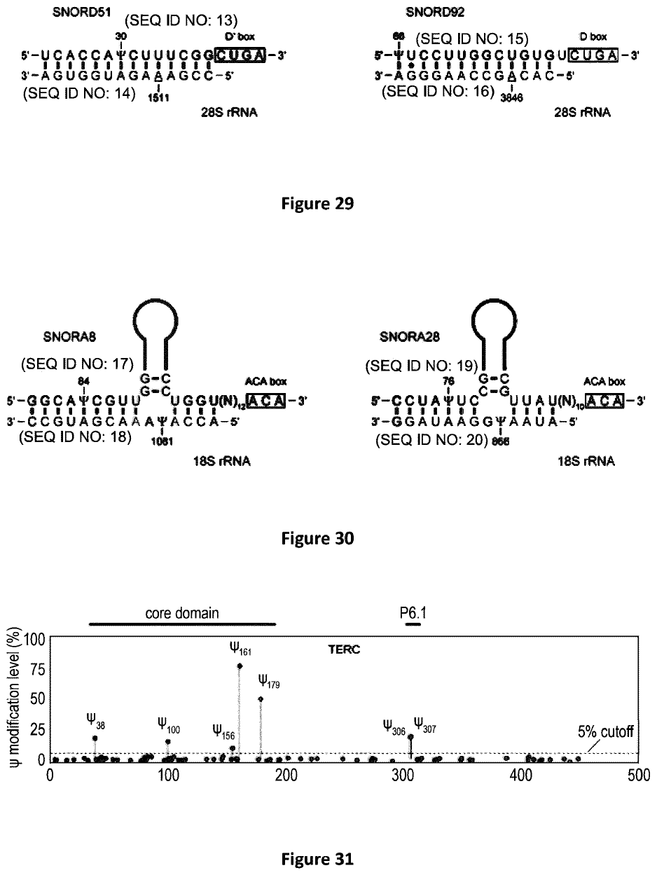

The ψ profile of yeast snoRNA has been revealed through Pseudo-seq and ψ-seq, yet it remains relatively unexplored in human snoRNA. Using BACS, 282 ψ sites were detected in snoRNA from HeLa cells, including those previously identified by ψ-seq and BID-seq (see FIGS. 23, 24a and 24b). Analysis revealed the presence of 192, 62, and 28 ψ sites in box C/D snoRNAs, box H/ACA snoRNAs, and small Cajal body-specific RNAs (scaRNAs), respectively. Remarkably, all three types of snoRNAs exhibited a substantial number of highly modified ψ sites (see FIGS. 25 and 26). Furthermore, W sites observed in box C/D snoRNAs displayed enrichment in the 5′-upstream regions of box D′ and the 3′-downstream regions of box C′, while ψ sites in box H/ACA snoRNAs were enriched in the 5′-upstream regions of box H and ACA (see FIGS. 27 and 28). These patterns implied a potential role for ψ in mediating interactions between snoRNAs and their targets. Indeed, a subset of ψ sites identified in box C/D and box H/ACA snoRNAs were also located in the predicted guide regions, which was in accordance with the ψ-seq results (see FIGS. 29 and 30).

In addition, human telomerase RNA component (TERC) shares similar characteristics with snoRNAs, as it contains a conserved box H/ACA scaRNA domain at the 3′-end. Upon BACS treatment, 7 ψ sites in TERC from HeLa cells, 4 of which were putative ψ sites previously discovered through CMC-based primer extension approach (see FIGS. 23 and 31). In particular, all 3 novel ψ sites (ψ₃₈/ψ₁₀₀/ψ₁₅₅), together with the known ψ₁₆₁ and ψ₁₇₉ sites, were found in the core domain of TERC. This observation suggested the potential involvement of ψ in stabilizing the TERC structure, similar to the scenario that has been demonstrated for ψ₃₀₆ and ψ₃₀₇ within the P6.1 loop of TERC.

### Example 7: a Comprehensive ψ Map of Human tRNA

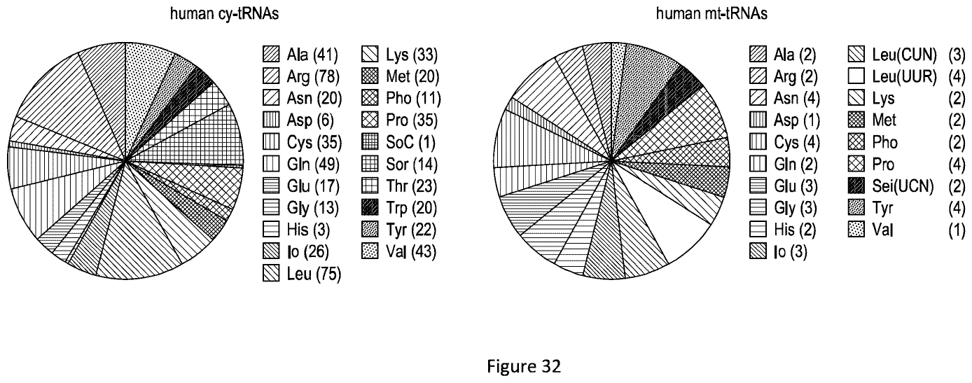

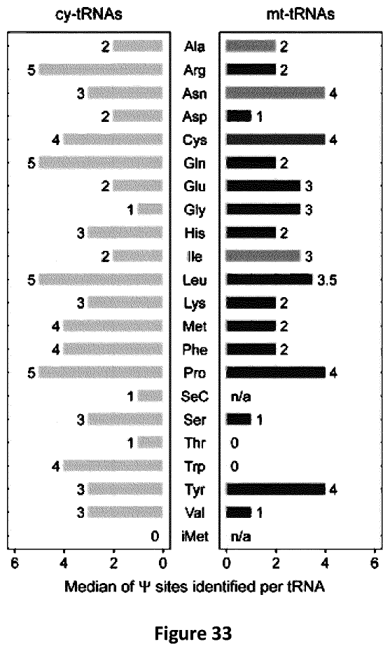

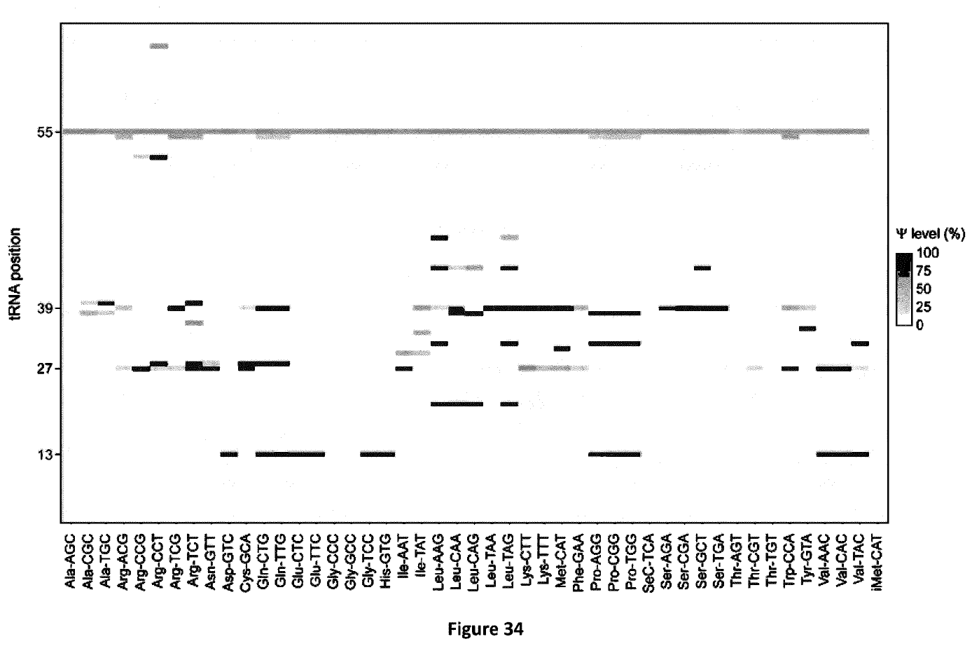

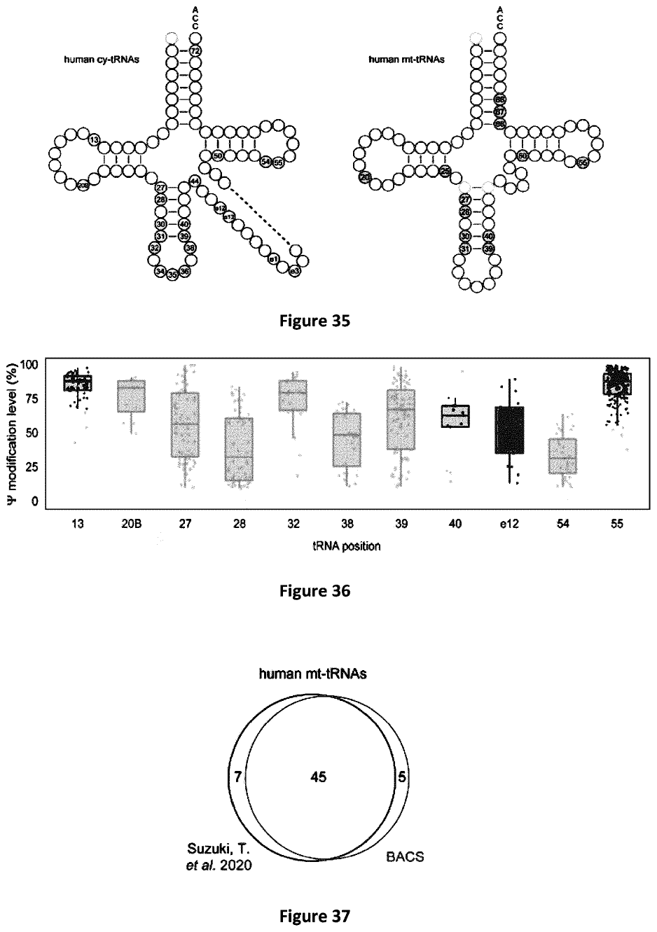

ψ is one of the most fundamental and prevalent modifications in human tRNA. However, given that most of tRNA species are extensively modified and highly structured, quantitative profiling of ψ in tRNA remains challenging by CMC- or BS-based methods. BACS offers a better solution to this problem, since mutation signals induced by BACS would not be significantly influenced by RT blocks or other intrinsic mismatches. BACS was applied to tRNA from HeLa cells and successfully detected 625 high-confidence ψ sites in cytosolic tRNAs (cy-tRNAs) (see FIG. 32). The number of ψ sites identified per cy-tRNA varied among different isoacceptor families (see FIG. 33). In cy-tRNAs, ψ sites were predominantly located at highly conserved positions, including position 13, 27-28, 38-40, and 55, while ψ at other positions were limited to specific types of cy-tRNAs (see FIG. 34). An integrated view of the ψ profile of human cy-tRNAs was then summarized based on the canonical tRNA numbering system (see FIG. 35). Subsequently, the ψ modification level at each tRNA position is compared, providing valuable insights into the properties of the corresponding PUS enzymes (see FIG. 36). Notably, position 55 emerged as the most frequently and highly modified ψ site in cy-tRNAs, which is mainly installed by TRUB1. Moreover, position 13, known as a PUS7 target, also displayed a high level of ψ modification. In contrast, the modification levels of PUS1 targets (position 27-28) and PUS3 targets (position 38-40) exhibited considerable variations. Further confirmation of the responsible PUS enzymes for other positions will be important to fully understand the diverse and specific patterns of ψ modifications in human cy-tRNAs.

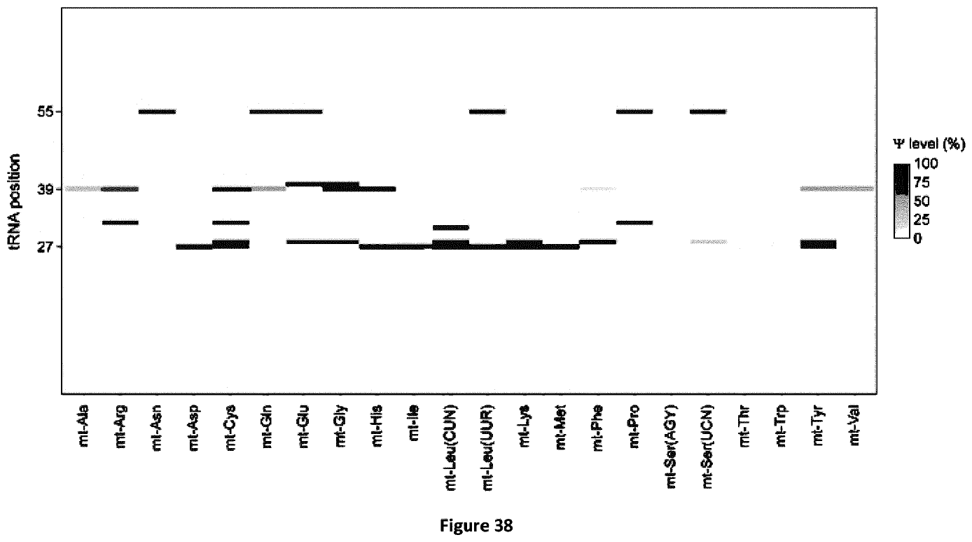

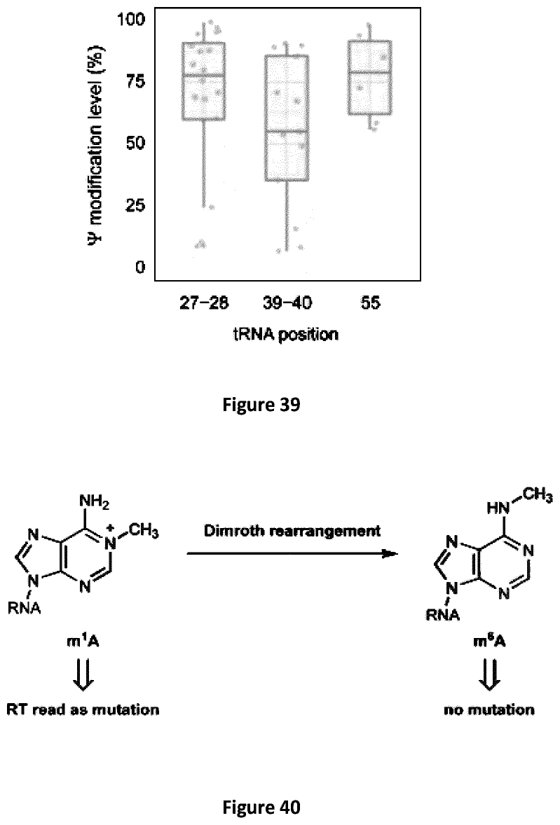

Using BACS, 50 ψ sites were also detected in human mitochondrial tRNAs (mt-tRNAs), which was highly consistent with the published dataset (see FIGS. 32, 33 and 37). Three reported ψ sites (including ψ₃₈ in mt-tRNA^{Ala}, ψ₅₅ in mt-tRNA^{Met}, and ψ₃₈ in mt-tRNA^{Pro}) were not characterized as high-confidence sites due to their low modification levels (<5%, see FIG. 38). Although BACS clearly showed higher resolution than CMC- and BS-based methods for tRNA ψ profiling, ψ₂₀ in mt-tRNA^{Leu(CNN)} and ψ₂₅ in mt-tRNA^{Asn} detected by BACS were not located at common positions and still requires further validation (see FIG. 35). Overall, human mt-tRNAs were pseudouridylated to a less extent compared with cy-tRNAs (see FIGS. 34, 37, 38 and 39).

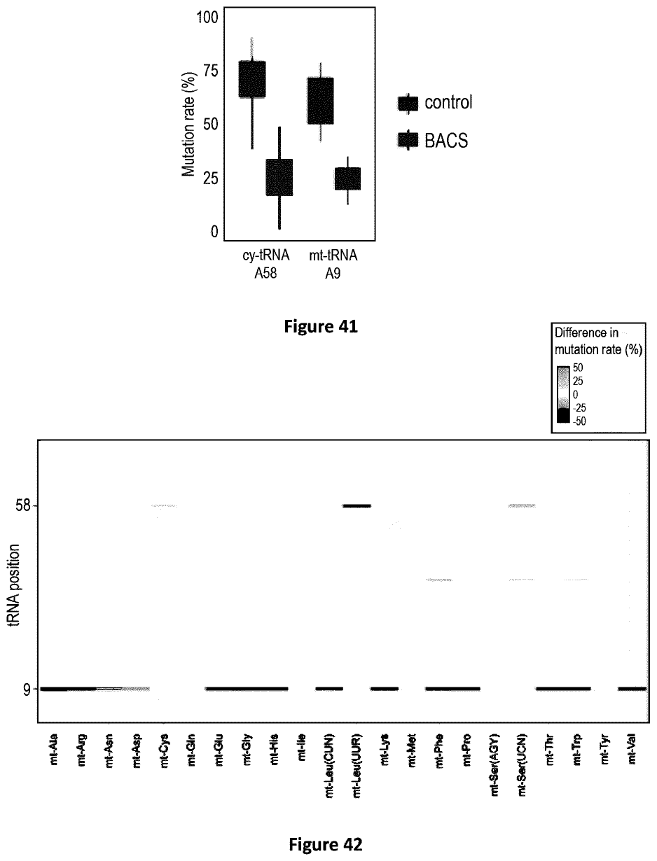

Similar to RBS-seq, BACS would also induce Dimroth rearrangement of N1-methyladenosine (m¹A) to N⁶-methyladenosine (m⁶A) and therefore could potentially detect m¹A together with ψ (see Supplementary FIG. 6a). As expected, a significant reduction of m¹A mutation signals was observed at tRNA position 58 (for cy-tRNAs) and 9 (for mt-tRNAs) after BACS treatment, which was comparable to the efficiency of demethylase (see FIGS. 41 and 42). These results suggest that BACS could be a powerful tool to study multiple modifications simultaneously in tRNA.

### Example 8: Profiling and Quantification of ψ in HeLa mRNA

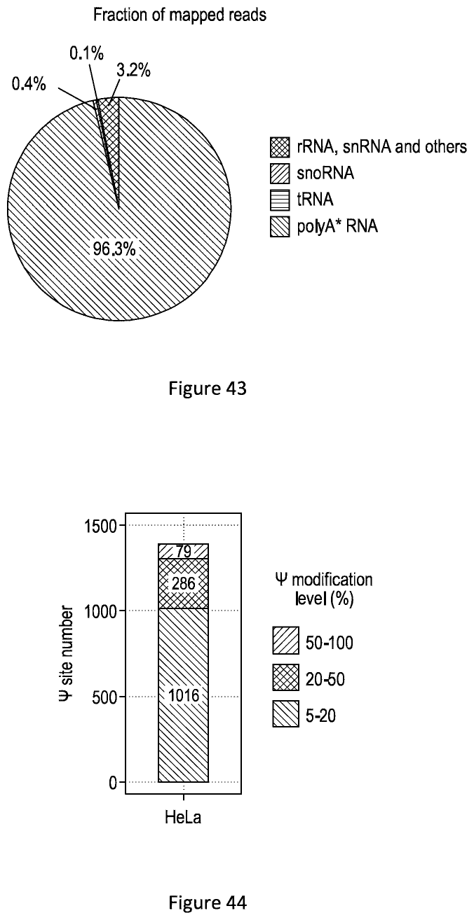

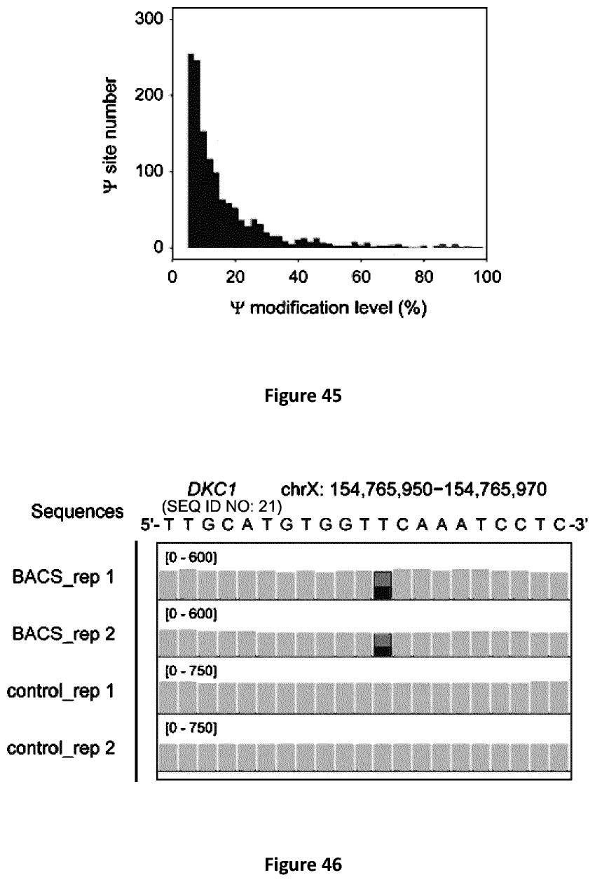

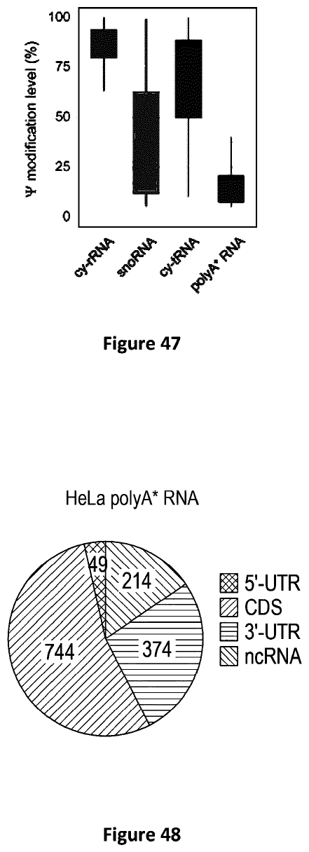

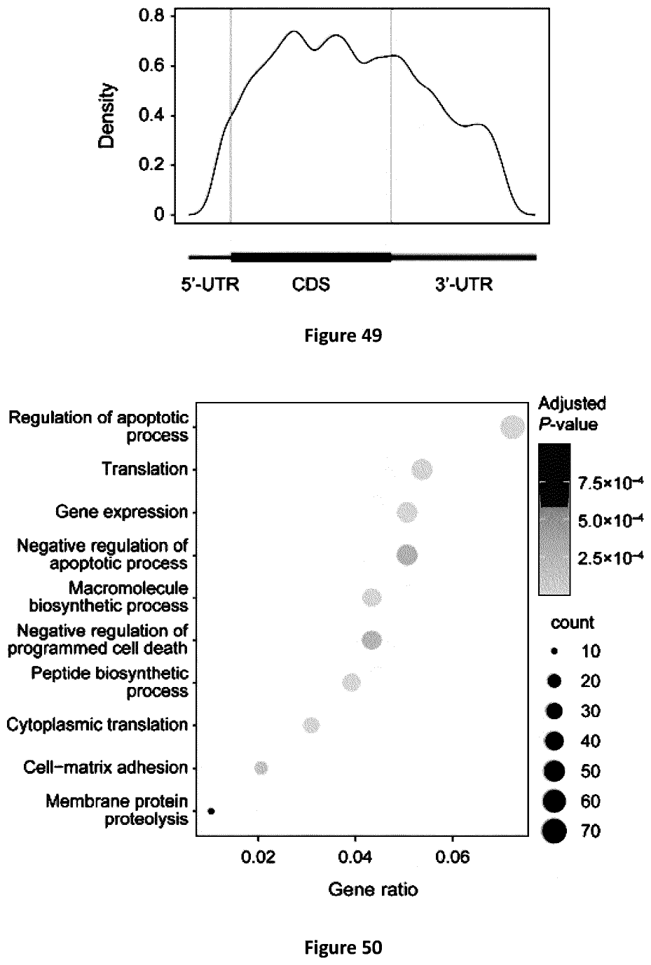

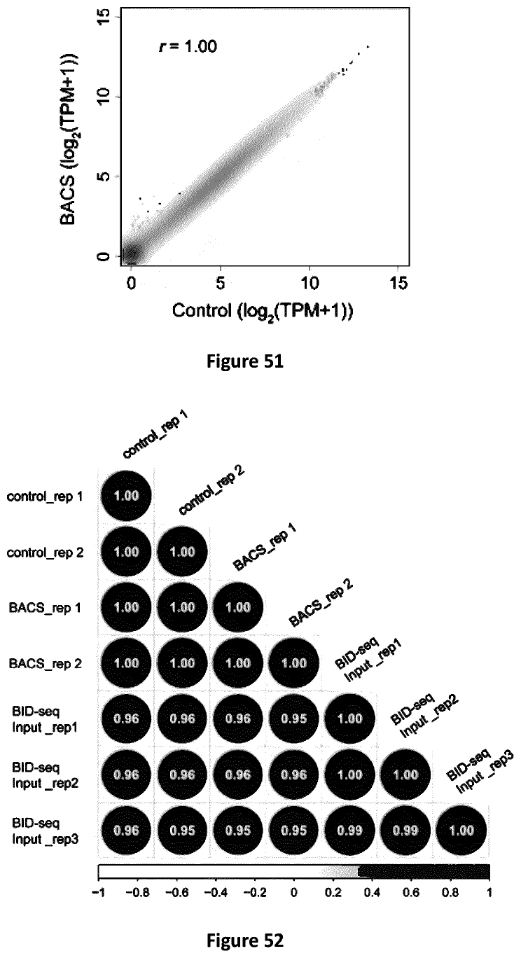

After successfully applying BACS to various types of ncRNAs, it was used to map and quantify ψ modifications in HeLa mRNA. Given the high abundance of ψ in rRNA, snRNA, snoRNA, and tRNA, the fraction of reads mapped to them was examined in the polyA-tailed RNA samples to evaluate the efficiency of enrichment. Only a small proportion of reads (3.7%) was mapped to these ncRNAs (see FIG. 43). With the remaining reads, a total of 1381 ψ sites were mapped in HeLa polyA-tailed RNA (see FIG. 44). The majority of these ψ sites exhibited low modification levels (<20%), while only a limited number of ψ sites displayed high levels of modification (>50%) (see FIGS. 44 and 45). A representative highly modified ψ site in DKC1 was presented, as demonstrated by high U-to-C mutation signals in BACS libraries and low backgrounds in control libraries (see FIG. 46). In contrast to the aforementioned ncRNAs, the ψ modification level in polyA-tailed RNA was significantly lower (see FIG. 47). Among the 1381 ψ sites, 1167 and 214 of them were located in mRNA and ncRNA (excluding rRNA, snRNA, snoRNA, and tRNA), respectively (see FIG. 48). Within mRNA, ψ was enriched in the coding sequence (CDS) and 3′-untranslated region (3′-UTR), while it was relatively depleted in the 5′-untranslated region (5′-UTR), consistent with previous findings (see FIGS. 48 and 49). The gene ontology (GO) analysis revealed that ψ-modified mRNA was enriched in functions such as translation and regulation of apoptotic process (see FIG. 50). Importantly, BACS could simultaneously provide the mRNA expression levels while mapping ψ, which showed strong correlation with control libraries (Pearson's r=1.00) and BID-seq input libraries (Pearson's r=0.95-0.96), suggesting minimal RNA degradation induced by BACS (see FIGS. 51 and 52).

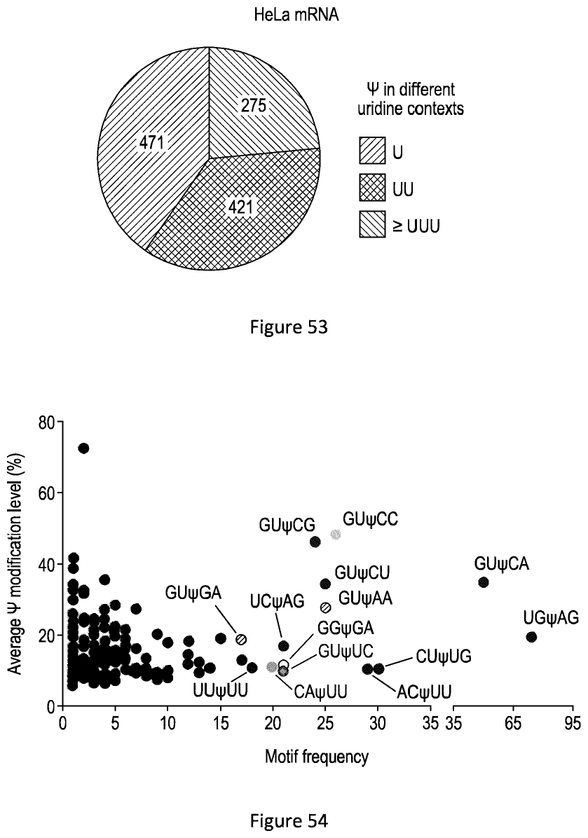

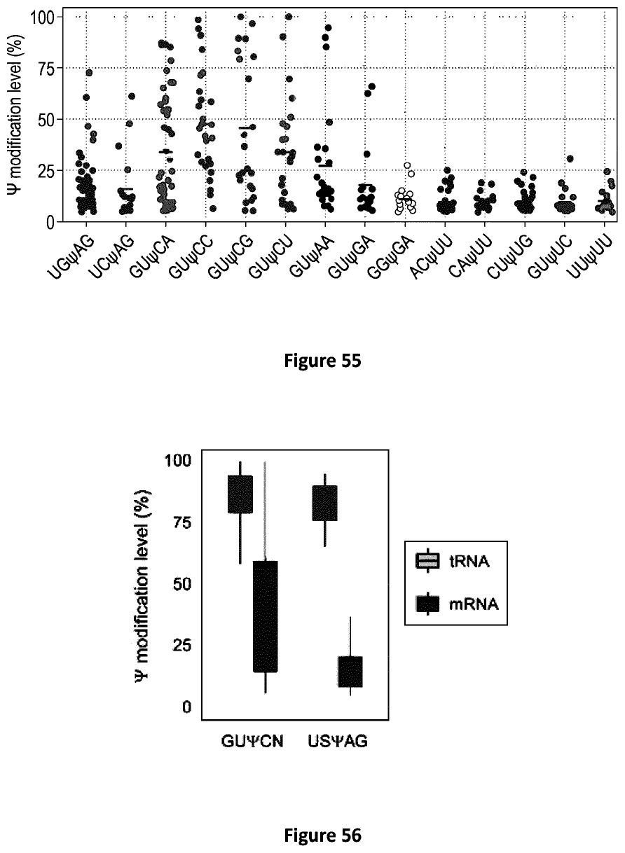

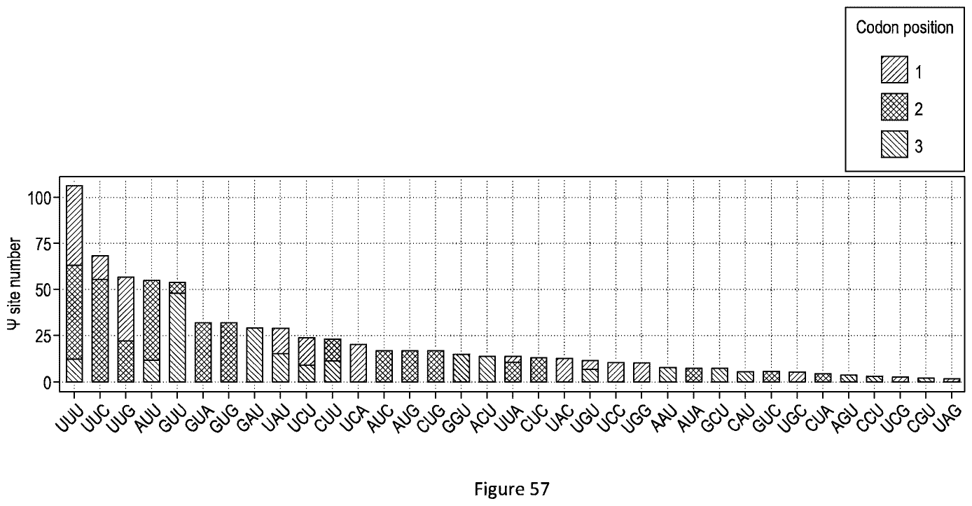

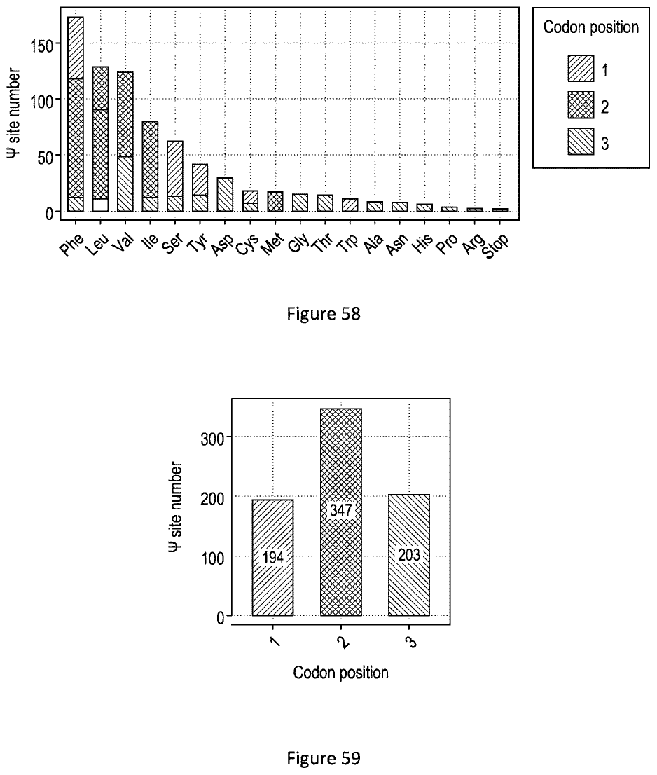

Next, the sequence contexts of ψ in HeLa mRNA was analyzed. First, analysis indicated that the majority of ψ sites (59.6%) were located in consecutive uridine sequences (see FIG. 53). These positions could not be precisely determined through BS-based methods, further highlighting the advantage of BACS. Benefiting from the high-resolution signals of BACS, ψ was predominantly found enriched in USψAG (S═C or G) and GUψCN (N=A, C, G or U) motifs, corresponding to the previously identified PUS7 and TRUB1 motif, respectively (see FIG. 54). In addition, it was also observed that ψ tends to enrich in those motifs containing multiple consecutive uridines, such as CUψUG, ACψUU, and even UUψUU. The stoichiometry of ψ within these motifs were also compared, demonstrating that GUψCN exhibited a relatively high modification level (see FIG. 55). However, these potential TRUB1 targets in mRNA were significantly less modified than their counterparts in cy-tRNAs, which was similarly observed for the putative PUS7 targets (see FIG. 56). These results suggested that mRNA may not be the primary substrate of these stand-alone PUS enzymes. Furthermore, from analysis of the codon preference of ψ in mRNA, ψ was enriched in those codons containing consecutive uridines, such as UUY (Y=C or U), UUG, AUU, and GUU, which encoded phenylalanine (Phe), leucine (Leu), isoleucine (lie), and valine (Val), respectively (see FIGS. 57 and 58). Within codons, ψ was mainly located in the second position (see FIG. 59). A single ψ site positioned in the start codon (AUG) was observed, whilst 2 sites were found in the stop codon (UAG). These may promote stop codon readthrough.

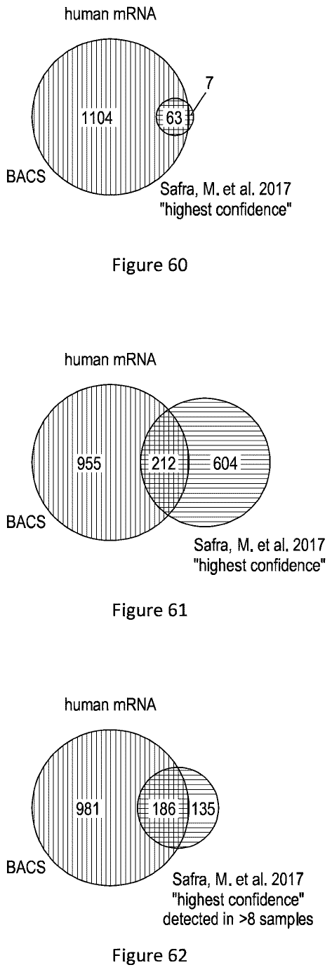

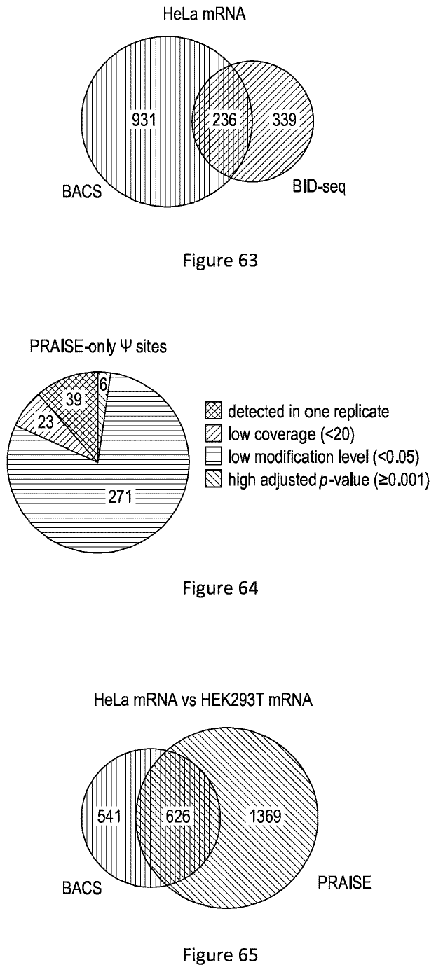

To further evaluate the performance of BACS, a thorough comparison was made of the identified mRNA ψ sites with published datasets. First, BACS was compared with a recent dataset (Safra, M., et al., Genome Res. 27, 393-406 (2017)) which consolidated three CMC-based methods. Remarkably, BACS accurately identified 63 out of 70 ψ sites (90.0%) listed in the “highest confidence” category (see FIG. 60). However, a strong overlap between BACS and the “high confidence” list was achieved only when considering ψ sites consistently detected across multiple samples (>8) (186 out of 321 ψ sites, 57.9%, (see FIGS. 61 and 62). BACS was further compared with two recently developed BS-based methods. Compared to CMC-based approaches, BACS demonstrated a better overlap with BID-seq results, as expected (236 out of 575 ψ sites, 41.0%, (see FIG. 63).

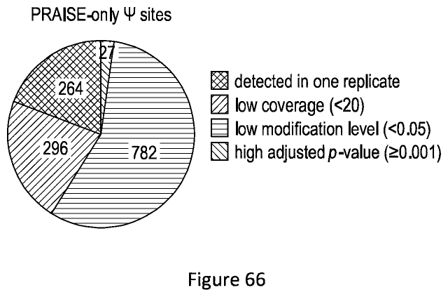

Most of the sites exclusive to BID-seq dataset displayed low modification levels in our BACS libraries (see FIG. 64). When compared with PRAISE, 626 of 1995 ψ sites (31.4%) showed an overlap with BACS results (see FIG. 65). Similarly, the majority of PRAISE-only ψ sites were lowly modified in our dataset (see FIG. 66). Regarding the 6 ψ sites identified in mitochondrial mRNAs (mt-mRNAs) by BACS, 4, 2, and 3 of them have also been detected by Pseudo-seq, BID-seq, and PRAISE, respectively. Potentially, the degree of overlap between different methods may be influenced by the differences in sequencing depths and the distinct bioinformatics pipelines used for analysis (for example, mapping to the genome or directly to the transcriptome).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of modifying pseudouridine comprising reacting the pseudouridine with a Michael Addition acceptor,
wherein the Michael Addition acceptor is a compound according to Formula (I): 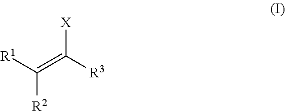
wherein:
X is independently selected from halo, tosyl, mesyl, and triflyl;
R¹ is independently selected from H, C₁-C₄-alkyl, C₁-C₄-haloalkyl, and C₀-C₄-alkylene-R^{1a}, wherein R^{1a} is independently selected from C₃-C₆ cycloalkyl, phenyl, and 4- to 6-membered heterocyclyl; wherein where R^{1a} is C₃-C₆ cycloalkyl or 4- to 6-membered heterocyclyl, R^{1a} is optionally substituted with from 1 to 4 R^{1b}, and where R^{1a} is phenyl, R^{1a} is optionally substituted with from 1 to 4 R^{1c};
R² is independently selected from H, C₁-C₄-alkyl, C₁-C₄-haloalkyl, and C₀-C₄-alkylene-R^{2a}, wherein R^{2a} is independently selected from C₃-C₆ cycloalkyl, phenyl, and 4- to 6-membered heterocyclyl; wherein where R^{2a} is C₃-C₆ cycloalkyl or 4- to 6-membered heterocyclyl, R^{2a} is optionally substituted with from 1 to 4 R^{2b}, and where R^{2a} is phenyl, R^{2a} is optionally substituted with from 1 to 4 R^{2c};
R³ is independently selected from —C(O)OR^{3a}, —C(O)R^{3a}, —C(O)NR^{3b}R^{3b}, —CN, —NO₂, —S(O)₂OR^{3a}, —S(O)₂R^{3a}, —S(O)₂NR^{3b}R^{3b}, and 5- to 10-membered heteroaryl, wherein where R³ is 5- to 10-membered heteroaryl, R³ is optionally substituted with from 1 to 4 R^{3e};
R^{3a} is independently selected from H, C₁-C₆-alkyl, C₁-C₆-haloalkyl, and C₀-C₆-alkylene-R^{3c}; wherein where R^{3a} is alkyl or haloalkyl, R^{3a} is optionally substituted with a group selected from —N₃, —C≡CH, Dibenzocyclooctynol (DIBO), Aza-dibenzocyclooctyne (DBCO), Bicyclononyne (BCN), trans-Cyclooctene (TCO), and Tetrazine;
R^{3b} is independently selected from H, C₁-C₆-alkyl, C₁-C₆-haloalkyl, and C₀-C₆-alkylene-R^{3c}; wherein where R^{3b} is alkyl or haloalkyl, R^{3b} is optionally substituted with a group selected from —N₃, —C≡CH, Dibenzocyclooctynol (DIBO), Aza-dibenzocyclooctyne (DBCO), Bicyclononyne (BCN), trans-Cyclooctene (TCO), and Tetrazine;
or wherein two R^{3b} groups, together with the nitrogen atom to which they are attached form a 5- or 6-membered heterocycloalkyl group, optionally substituted with from 1 to 4 R^{3d};
R^{3c} is independently selected from C₃-C₆ cycloalkyl, phenyl, and 4- to 6-membered heterocyclyl; wherein where R^{3c} is C₃-C₆ cycloalkyl or 4- to 6-membered heterocyclyl, R^{3c} is optionally substituted with from 1 to 4 R^{3d}, and where R^{3c} is phenyl, R^{3c} is optionally substituted with from 1 to 4 R^{3e};
R^{1b} and R^{2b} are each independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
R^{1c} and R^{2c} are each independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
R^{3d} is independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, C₁-C₄-haloalkyl, —N₃, —C≡CH, Dibenzocyclooctynol (DIBO), Aza-dibenzocyclooctyne (DBCO), Bicyclononyne (BCN), trans-Cyclooctene (TCO), and Tetrazine;
R^{3e} is independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl, —N₃, —C≡CH, Dibenzocyclooctynol (DIBO), Aza-dibenzocyclooctyne (DBCO), Bicyclononyne (BCN), trans-Cyclooctene (TCO), and Tetrazine;
R⁴ is independently at each occurrence selected from H and C₁-C₄-alkyl; or where two R⁴ groups are attached to the same nitrogen, those two R⁴ groups together with the nitrogen atom to which they are attached optionally form a 5- to 6-membered-heterocycloalkyl group optionally substituted with from 1 to 4 R⁵; and
R⁵ are each independently at each occurrence selected from ═O, ═S, halo, nitro, cyano, C₁-C₄-alkyl, and C₁-C₄-haloalkyl.

2. A method of tagging or labelling pseudouridine in a sample, comprising reacting at least a portion of the sample with a Michael Addition acceptor, wherein the Michael Addition acceptor is a compound according to Formula (I): 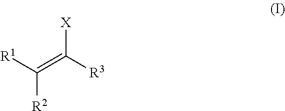
wherein:
X is independently selected from halo, tosyl, mesyl and triflyl;
R¹ is independently selected from H, C₁-C₄-alkyl, C₁-C₄-haloalkyl, and C₀-C₄-alkylene-R^{1a}, wherein R^{1a} is independently selected from C₃-C₆ cycloalkyl, phenyl, and 4- to 6-membered heterocyclyl; wherein where R^{1a} is C₃-C₆ cycloalkyl or 4- to 6-membered heterocyclyl, R^{1a} is optionally substituted with from 1 to 4 R^{1b}, and where R^{1a} is phenyl, R^{1a} is optionally substituted with from 1 to 4 R¹⁰;
R² is independently selected from H, C₁-C₄-alkyl, C₁-C₄-haloalkyl, and C₀-C₄-alkylene-R^{2a}, wherein R^{2a} is independently selected from C₃-C₆ cycloalkyl, phenyl, and 4- to 6-membered heterocyclyl; wherein where R^{2a} is C₃-C₆ cycloalkyl or 4- to 6-membered heterocyclyl, R^{2a} is optionally substituted with from 1 to 4 R^{2b}, and where R^{2a} is phenyl, R^{2a} is optionally substituted with from 1 to 4 R^{2c};
R³ is independently selected from —C(O)OR^{3f}, —C(O)R^{3f}, —C(O)NR^{3e}R^{3f}, —S(O)₂OR^{3f}, —S(O)₂R^{3f}, —S(O)₂NR^{3e}R^{3f};
R^{3f} is a linker covalently linked to an affinity tag or imaging probe;
R^{3g} is independently selected from H, C₁-C₆-alkyl, and C₁-C₆-haloalkyl; R^{1b} and R^{2b} are each independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
R^{1c} and R^{2c} are each independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
R⁴ is independently at each occurrence selected from H and C₁-C₄-alkyl; or where two R⁴ groups are attached to the same nitrogen, those two R⁴ groups together with the nitrogen atom to which they are attached optionally form a 5- to 6-membered-heterocycloalkyl group optionally substituted with from 1 to 4 R⁵; and
R⁵ are each independently at each occurrence selected from ═O, ═S, halo, nitro, cyano, C₁-C₄-alkyl, and C₁-C₄-haloalkyl.

3. A method as claimed in claim 2, wherein the affinity tag is selected from the group comprising biotin, FLAG tag, His-tag, HA tag, Strep-tag, Avi-tag, GST, c-myc-tag, V5-tag, E-tag, S-tag, SBP-tag, poly (Glu)-tag, calmodulin tag.

4. A method as claimed in claim 2 or claim 3, wherein the linker is a flexible linker, a cleavable linker; optionally a photocleavable linker.

5. A method as claimed in claim 4, wherein the linker is selected from (a) a polyethylene glycol; (b) a peptide; (c) a nucleic acid; or (d) an oligosaccharide.

6. A method as claimed in claim 2, wherein the imaging probe is selected from the group comprising fluorescent moieties, radionuclides and metal complexes.

7. A method as claimed in claim 6, wherein the imaging probe is (a) a fluorophore selected from the group comprising a fluorescein, a rhodamine, BIODIPY, an Alexa fluor, a Cy dye or an ATTO dye; (b) a lanthanide complex; or (c) a radionuclide complex.

8. A method of isolating RNA comprising pseudouridine from a sample, comprising reacting the sample with a Michael Addition acceptor according to a method of any of claims 2 to 5, and then contacting the sample with a substrate comprising the binding partner to the affinity tag.

9. A method as claimed in claim 8, wherein the affinity tag is biotin and the binding partner is avidin or streptavidin.

10. A method of visualising pseudouridine in a sample comprising RNA, comprising reacting the sample with a Michael Addition acceptor according to a method of any of claims 2, 6 or 7, and subjecting the sample to a visualization procedure selected from optical observation, microscopical observation and image capture.

11. A method of determining the presence and sequence location of pseudouridine comprised in a sample of RNA comprising:
(a) reacting at least a portion of the sample with a Michael Addition acceptor, wherein the Michael Addition acceptor is a compound according to Formula (I): 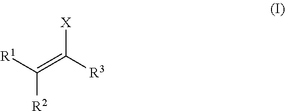
wherein:
X is independently selected from halo, tosyl, mesyl and triflyl;
R¹ is independently selected from H, C₁-C₄-alkyl, C₁-C₄-haloalkyl, and C₀-C₄-alkylene-R^{1a}, wherein R^{1a} is independently selected from C₃-C₆ cycloalkyl, phenyl, and 4- to 6-membered heterocyclyl; wherein where R^{1a} is C₃-C₆ cycloalkyl or 4- to 6-membered heterocyclyl, R^{1a} is optionally substituted with from 1 to 4 R^{1b}, and where R^{1a} is phenyl, R^{1a} is optionally substituted with from 1 to 4 R^{1c};
R² is independently selected from H, C₁-C₄-alkyl, C₁-C₄-haloalkyl, C₀-C₄-alkylene-R^{2a}, wherein R^{2a} is independently selected from C₃-C₆ cycloalkyl, phenyl, and 4- to 6-membered heterocyclyl; wherein where R^{2a} is C₃-C₆ cycloalkyl or 4- to 6-membered heterocyclyl, R^{2a} is optionally substituted with from 1 to 4 R^{2b}, and where R^{2a} is phenyl, R^{2a} is optionally substituted with from 1 to 4 R^{2c};
R³ is independently selected from —C(O)OR^{3a}, —C(O)R^{3a}, —C(O)NR^{3b}R^{3b}, —CN, —NO₂, —S(O)₂OR^{3a}, —S(O)₂R^{3a}, —S(O)₂NR^{3b}R^{3b}, and 5- to 10-membered heteroaryl, wherein where R³ is 5- to 10-membered heteroaryl, R³ is optionally substituted with from 1 to 4 R^{3e};
R^{3a} is independently selected from H, C₁-C₆-alkyl, C₁-C₆-haloalkyl, and C₀-C₆-alkylene-R^{3c};
R^{3b} is independently selected from H, C₁-C₆-alkyl, C₁-C₆-haloalkyl, and C₀-C₆-alkylene-R^{3c};
or wherein two R^{3b} groups, together with the nitrogen atom to which they are attached form a 5- or 6-membered heterocycloalkyl group, optionally substituted with from 1 to 4 R^{3d};
R^{3c} is independently selected from C₃—C cycloalkyl, phenyl, and 4- to 6-membered heterocyclyl; wherein where R^{3c} is C₃—C cycloalkyl or 4- to 6-membered heterocyclyl, R^{3c} is optionally substituted with from 1 to 4 R^{3d}, and where R^{3c} is phenyl, R³⁰ is optionally substituted with from 1 to 4 R^{3e};
R^{1b}, R^{2b}, and R^{3d} are each independently at each occurrence selected from ═O, ═S, halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
R^{1c}, R^{2c}, and R^{3e} are each independently at each occurrence selected from halo, nitro, cyano, C(O)OR⁴, C(O)R⁴, C(O)NR⁴R⁴, C₁-C₄-alkyl, C₂-C₄-alkenyl, C₂-C₄-alkynyl, and C₁-C₄-haloalkyl;
R⁴ is independently at each occurrence selected from H and C₁-C₄-alkyl; or where two R⁴ groups are attached to the same nitrogen, those two R⁴ groups together with the nitrogen atom to which they are attached optionally form a 5- to 6-membered-heterocycloalkyl group optionally substituted with from 1 to 4 R⁵; and
R⁵ are each independently at each occurrence selected from ═O, ═S, halo, nitro, cyano, C₁-C₄-alkyl, and C₁-C₄-haloalkyl;
(b) (i) sequencing the RNA; or (ii) reverse transcribing the RNA resulting from step (a) to provide cDNA and amplifying the cDNA;
(c) sequencing the DNA of step (b)(ii);
(d) comparing the DNA sequence of step (c) with a reference DNA sequence to identify the sequence positions of guanine (G) in the sequence which are adenine (A) in the reference sequence, the position of G in the DNA sequence being the positions of a pseudouridine in the corresponding RNA sequence.

12. A method as claimed in claim 11, wherein the reference sequence is obtained from a separate portion of the RNA sample which is not subjected to Michael Addition reaction of step (a), but which is sequenced according to step (b)(i); or reverse transcribed according to step (b)(ii) and sequenced according to step (c).

13. A method as claimed in claim 11 or claim 12, wherein the amplification of cDNA employs an isothermal method of amplification; wherein the isothermal method of amplification is selected from polymerase chain reaction (PCR) strand-displacement amplification (SDA), rolling-circle amplification (RCA), whole-genome amplification (WGA), loop-mediated isothermal amplification (LAMP), helicase-dependent amplification (HDA), and multiple displacement amplification (MDA); optionally wherein a one-step RT-PCT is used.

14. A method as claimed in any of claims 11 to 13, wherein the reverse transcription of step (b) uses a reverse transcriptase enzyme; optionally selected from: Maxima H-, SuperScript II, SuperScript III, SuperScrupt IV, ProtoScript II, SMARTScribe, PrimeScript II, HiScript III, MMLV, AMV, TGIRT-III or recombinant HIV; preferably Maxima H-, SuperScript IV and TGIRT-III; more preferably Maxima H-.

15. A method as claimed in any preceding claim, wherein X is selected from Br, Cl, and I.

16. A method as claimed in any preceding claim, wherein at least one of R¹ and R² is H; optionally wherein both R¹ and R² are H.

17. A method as claimed in any preceding claim, wherein R³ is independently selected from —C(O)OR^{3a}, —C(O)R^{3a}, —C(O)NR^{3b}R^{3b}, and —CN.

18. A method as claimed in any preceding claim, wherein R³ is —C(O)NR^{3b}R^{3b}, optionally wherein R³ is —C(O)NH₂.

19. A method as claimed in any of claims 1 to 18, wherein the compound according to Formula (I) is selected from:

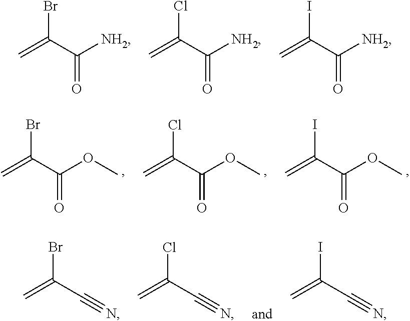

20. A method as claimed in any preceding claim, wherein the Michael Addition reaction is performed at a pH in the range of from about 7.0 to about 9.5.

21. A method as claimed in any preceding claim, wherein the Michael Addition acceptor is present at a concentration in the range from about 10 mM to about 2M; preferably in the range from about 100 mM to about 500 mM; more preferably about 250 mM.

22. A method as claimed in any preceding claim, wherein the Michael Addition reaction is performed at a temperature in the range of from about 25° C. to about 95° C.; preferably from about 65° C. to about 95° C.; more preferably about 85° C.

23. A method as claimed in any preceding claim, wherein the Michael Addition reaction is performed at a temperature of about 70° C. or greater for a time in the range of from about 5 minutes to about 2 hours; or at a temperature of about 70° C. or less for a time in the range of from about 2 hours to about 16 hours.

24. A method as claimed in any preceding claim, wherein the RNA molecule is selected from one or more of mRNA, tRNA, rRNA, snRNA, miRNA, lncRNA or circRNA.

25. A method as claimed in any preceding claim, wherein the RNA molecule is derived from a biological sample.

26. A kit for modifying a pseudouridine comprising:
(a) a solution comprising a Michael Addition acceptor as set forth in any of claims 1 to 7, or 15 to 19;
(b) instructions for reacting a sample comprising pseudouridine with the solution.

27. A kit for tagging or labelling pseudouridine comprised in RNA, comprising:
(a) a solution comprising a Michael Addition acceptor as set forth in any of claims 2 or 15 to 19;
(b) instructions for reacting an RNA with the solution.

28. A kit for determining the presence of sequence location of pseudouridine comprised in RNA, comprising:
(a) a solution comprising a Michael Addition acceptor as set forth in any of claims 11 or 15 to 19;
(b) instructions for reacting an RNA with the solution.

29. A kit as claimed in any of claims 26 to 28, further comprising one or more buffers.

30. A kit as claimed in claim 28 or claim 29, further comprising a reverse transcriptase enzyme; optionally a reverse transcriptase selected from: Maxima H-, SuperScript II, SuperScript III, SuperScrupt IV, ProtoScript II, SMARTScribe, PrimeScript II, HiScript III, MMLV, AMV, TGIRT-III, recombinant HIV, Marathon reverse transcriptase or Induro reverse transcriptase; preferably Maxima H-, SuperScript IV and TGIRT-III; more preferably Maxima H-.

31. A kit as claimed in any of claims 28 to 30, further comprising a DNA polymerase; optionally a DNA polymerase selected from Taq DNA Polymerase, Bst DNA Polymerase or Bsu DNA Polymerase.


SEQUENCE LISTING (21 sequences)

SEQ ID NO: 1 - skipped

SEQ ID NO: 2 - RNA, 10 nt, synthetic construct
  source 1..10: mol_type = other RNA
  misc_feature 1..10: note = Control oligonucleotide
tactgtagct

SEQ ID NO: 3 - DNA, 89 bp, synthetic construct
  source 1..89: mol_type = other DNA
  misc_feature 1..89: note = Template DNA sequence
gttgtctttgccttcgcttcggtcctcgatttctgttgttgtaccgttggtttcgttgtggtgtgttctccctatagtgagtcgtatta

SEQ ID NO: 4 - RNA, 72 nt, synthetic construct
  source 1..72: mol_type = other RNA
  modified_base 30: mod_base = p
  modified_base 44: mod_base = p
  misc_feature 1..72: note = RNA sequence
gggagaacacaccacaacgaaaccaacggnacaacaacagaaancgaggaccgaagcgaaggcaaagacaac

SEQ ID NO: 5 - RNA, 41 nt, unidentified
  source 1..41: mol_type = rRNA
  misc_feature 1..41: note = Sequence
cccgaagcgtttactttgaaaaaattagagtgttcaaagca

SEQ ID NO: 6 - RNA, 31 nt, unidentified
  source 1..31: mol_type = rRNA
  misc_feature 1..31: note = Sequence
gcctagtgggccacttttggtaagcagaact

SEQ ID NO: 7 - RNA, 21 nt, unidentified
  source 1..21: mol_type = rRNA
  misc_feature 1..21: note = Sequence
gagtaactatgactctcttaa

SEQ ID NO: 8 - RNA, 41 nt, unidentified
  source 1..41: mol_type = rRNA
  misc_feature 1..41: note = Sequence
aaagctcgcttgatcttgattttcagtacgaatacagaccg

SEQ ID NO: 9 - RNA, 31 nt, unidentified
  source 1..31: mol_type = rRNA
  misc_feature 1..31: note = Sequence
cctgcggcttaatttgactcaacacgggaaa

SEQ ID NO: 10 - RNA, 31 nt, unidentified
  source 1..31: mol_type = rRNA
  misc_feature 1..31: note = Sequence
aatagggaacgtgagctgggtttagaccgtc

SEQ ID NO: 11 - RNA, 17 nt, unidentified
  source 1..17: mol_type = other RNA
  misc_feature 1..17: note = U6atac snRNA
ccccttgacaaggatgg

SEQ ID NO: 12 - RNA, 17 nt, unidentified
  source 1..17: mol_type = other RNA
  misc_feature 1..17: note = U4atac snRNA
  modified_base 9..10: mod_base = p
ccatccttnncttgggg

SEQ ID NO: 13 - RNA, 18 nt, unidentified
  source 1..18: mol_type = other RNA
  misc_feature 1..18: note = SNORD51
  modified_base 7: mod_base = p
tcaccanctttcggctga

SEQ ID NO: 14 - RNA, 14 nt, unidentified
  source 1..14: mol_type = rRNA
  misc_feature 1..14: note = 28S rRNA
ccgaaagatggtga

SEQ ID NO: 15 - RNA, 18 nt, unidentified
  source 1..18: mol_type = other RNA
  misc_feature 1..18: note = SNORD92
  modified_base 1: mod_base = p
ntccttggctgtgtctga

SEQ ID NO: 16 - RNA, 13 nt, unidentified
  source 1..13: mol_type = rRNA
  misc_feature 1..13: note = 28S rRNA
cacagccaaggga

SEQ ID NO: 17 - RNA, 32 nt, unidentified
  source 1..32: mol_type = other RNA
  misc_feature 1..32: note = SNORA8
  modified_base 5: mod_base = p
ggcancgttggcctggtnnnnnnnnnnnnaca

SEQ ID NO: 18 - RNA, 15 nt, unidentified
  source 1..15: mol_type = rRNA
  misc_feature 1..15: note = 18S rRNA
  modified_base 5: mod_base = p
accanaaacgatgcc

SEQ ID NO: 19 - RNA, 28 nt, unidentified
  source 1..28: mol_type = other RNA
  misc_feature 1..28: note = SNORA28
  modified_base 5: mod_base = p
cctantccgcgttatnnnnnnnnnnaca

SEQ ID NO: 20 - RNA, 13 nt, unidentified
  source 1..13: mol_type = rRNA
  misc_feature 1..13: note = 18S rRNA
  modified_base 5: mod_base = p
ataanggaatagg

SEQ ID NO: 21 - RNA, 21 nt, Homo sapiens
  source 1..21: mol_type = unassigned RNA
  misc_feature 1..21: note = HeLa polyA-tailed RNA
ttgcatgtggttcaaatcctc